(19) United States
(12) Patent Application Publication
(10) Pub. No.: US 2003/0021796 A1
(43) Pub. Date: Jan. 30, 2003

(54) Method of enhancing T cell immunity by selection of antigen specific T cells

(76) Inventors: Leszek Ignatowicz, Evans, GA (US); Piotr Kraj, Augusta, GA (US)

Correspondence Address: Charles P. Landrum, FULBRIGHT & JAWORSKI L.L.P., SUITE 2400, 600 CONGRESS AVENUE, AUSTIN, TX, 78701-3271, US

(21) Appl. No.: 10/137,745

(22) Filed: May 2, 2002

Related U.S. Application Data
(60) Provisional application No. 60/288,867, filed on May 4, 2001.

(51) Int. Cl. A61K 39/00; A61K 39/12; A61K 39/02; A61K 39/002

(52) U.S. Cl. 424/185.1; 424/186.1; 424/190.1; 424/191.1

(57) ABSTRACT

Disclosed is an in vivo system for the development of CD4⁺ T cells bearing class II MHC restricted TCR. The cells are induced by the administration of a positively selecting, soluble peptide. Following peptide delivery, double-positive CD4⁺CD8⁺ cells expressing this TCR differentiate into CD4⁺ cells in vivo, or in vitro in thymic organ cultures. This system facilitates the development of antigen-specific functional CD4⁺ T cells in a controlled manner, after administration of the peptide. The positively selected CD4⁺ T cells remain in the periphery for a prolonged time and respond to the appropriate antigenic challenge.

This application claims the priority of U.S. Provisional Application Ser. No. 60/288,867, filed May 4, 2001, the entire disclosure of which is specifically incorporated herein by reference. The government owns rights in the present invention pursuant to grant number NIH/NICHD RO1 HD36302-02 from the National Institutes of Health.

### BACKGROUND OF THE INVENTION

1. Field of the Invention

The present invention relates generally to the fields of immunology. More particularly, it concerns the ability to induce a population of naïve, antigen specific T cells in a host.

2. Description of Related Art

Thymic T cell ontogeny results in the generation of a mature T cell receptor repertoire that is able to recognize foreign antigens, yet these receptors, in normal conditions, do not react with self-tissues. Interaction of the antigen receptors on developing thymocytes with self MHC class I and class II molecules is the basis for positive and negative selection, and only thymocytes that have successfully completed the selection processes can leave the thymus. Various experimental approaches have revealed that thymocyte differentiation and selection are mutually interdependent, which makes it difficult to manipulate and recapitulate the development of such cells in a controlled manner. In particular, the specificity of interaction of TCR with MHC/peptide complexes has not been reproduced in vivo, and only partially in vitro.

T cell ontogeny is a multistep process resulting in the generation of mature peripheral CD4⁺ helper and CD8⁺ cytotoxic lymphocytes (Kisielow & von Boehmer, 1995). Most of the T cell ontogeny takes place in the thymus, where bone marrow derived precursors arrive and differentiate into α/β T cells as well as γ/β T cells, NK and thymic dendritic cells. Thymic ontogeny of α/β T cells is commonly divided into three major stages of double-negative (CD4^{-CD}8⁻), double-positive (CD4⁺CD8⁺), and single-positive (CD4⁺ or CD8⁺) cells based on the expression of CD4 and CD8 molecules. Thymocytes differentiate from the double-negative to double-positive stage by rearranging their TCR genes and expression of the TCR on the cell surface (Sebzda et al., 1999). At the same time, thymocytes undergo intensive clonal expansion so the vast majority of cells in the thymus are double-positive cells (Surh & Sprent, 1994). At this stage, thymocytes are subject to selection events that determine the TCR repertoire (Kisielow et al., 1988; Sha et al., 1988).

Interaction of TCRs with peptide/MHC complexes on the surface of thymic stromal cells produces a TCR repertoire that is tolerant to self-peptide/MHC and at the same time diversified enough to mount an effective immune response against almost all foreign antigens. Each thymocyte faces three outcomes of the selection process. First, the thymocyte may die by neglect if its TCR can not interact with peptide/MHC strongly enough to transduce the signal for positive selection. Second, thymocytes which have receptors interacting strongly with self-peptide/MHC receive a signal that causes apoptosis and they die in the process called negative selection. Third, thymocytes may undergo positive selection, survive and differentiate to single-positive CD4⁺ or CD8⁺ cells if their receptors interact weakly with peptide/MHC.

The processes of negative and positive selection ensure that T lymphocytes are tolerant to self-tissues and recognize antigens only as peptides presented by MHC molecules. The molecular mechanisms of the selection process have recently started to emerge (Ashton Rickardt et al., 1994). The expression of a number of genes (e.g., RAG, CD69, bcl-2, TCR, CD4, CD8) has been found to change in cells that undergo selection. However, the knowledge of the nature of the interaction between TCR and peptide/MHC in the thymus, and how it compares to the same kind of interaction between mature lymphocyte and antigen presenting cell, is still far from complete.

To date, some progress has been made to determine the properties of peptide ligands capable of inducing positive versus negative selection. Most of these experiments have been done on TCR MHC class I restricted transgenic thymocytes that developed in TAP and β2-microglobulin knockout mice with a reduced repertoire of self-peptides (Hogquist et al., 1994; Bill & Palmer, 1989; Nikolic-Zugic & Bevan, 1990). Because of the absence of selecting peptides in TAP or β2-microglobulin knockout mice, TCR transgenic cells develop only to the stage of double-positive thymocytes. Addition of exogenous peptides to TAP knockout fetal thymic organ cultures (FTOC) influenced selection of transgenic thymocytes. High concentration of strong agonist peptides was shown to induce negative selection, while low concentration, in some experiments induced positive selection (Hogquist et al., 1994; Hogquist et al., 1993). Peptide/MHC complexes that interacted very weakly with TCR induced positive selection (Hogquist et al., 1994; Bill & Palmer, 1989; Nikolic-Zugic & Bevan, 1990; Van Kaer et al, 1992). FTOC studies identified peptides that induced positive selection but when the same peptides were expressed in mice they did not induce positive selection (Bickoff et al., 1993). So far, no similar studies in FTOC were reported for class II restricted TCR.

It has been shown that virally-introduced antigenic peptide could induce positive selection of a small number of CD4⁺ cells (Miyazaki et al., 1996). In another study, MCC specific CD4⁺ cells developed in reaggregate cultures consisting of TCR transgenic thymocytes and thymic epithelial cell line (Hogquist et al., 1994; Nikolic-Zugic & Bevan, 1990; Van Kaer et al., 1992; Martin et al., 1996; Tourne et al., 1997). It is difficult to estimate the physiological significance of the latter finding, since reaggregate cultures lacked the critical cell population responsible for negative selection. Studies with class II restricted TCRs, however, a demonstrated peptide specific negative selection, antagonism of positive selection and selection into a CD8 single positive lineage (Kenty et al., 1998). This last finding may indicate that the signal that selected class II restricted T cells into the CD8 lineage was too weak.

Enhanced peptide diversity has been shown to improve the diversity of the TCR repertoire in experimental systems, which led authors to conclude that the previously defined “holes in this repertoire can be explained by the absence of an appropriate selecting self peptide.”The exogenous peptide could restore selection of the OT-I transgenic TCR in fetal thymic organ cultures (Stefanski et al., 2000). This result shows that “the T cell repertoire can be limited by a requirement for selecting peptides during development.”

It also has been found that newborns, in particular those born prematurely, are susceptible to infection with a variety of microorganisms. One of the reasons for higher disease susceptibility of these patients is the immaturity of the immune system. Despite the functional genetic mechanisms that generate T cell receptor repertoire its diversity remains limited contributing to the neonatal immunocompromised state (Schelonka et al., 1998).

Another paper states that “nonresponsivness to HBsAg vaccination is observed in 5-10% of vaccine recipients and is possibly caused by defect in the T helper compartment” (Hohler et al., 1998). This phenomenon may be related to the ‘hole’ in the repertoire of regulatory or helper T cells. The TCR repertoire diversity was notably decreased in patients receiving bone marrow transplants. These patients could benefit from therapy aimed at promoting the development and selection of T cells specific for particular antigens (Godthelp et al., 1999).

The production of thymocytes in adult thymus is diminished; however, it is believed that only quantitative changes affect thymopoesis in adult thymi (Ginaldi et al., 1999; Poulin et al., 1999). This makes adult individuals amenable for the therapy aimed at increasing the frequency of T cells with desired specificity by influencing thymic development. The need for modification of TCR repertoire post-natally is found in clinical literature that calls for the development of new clinical strategies that allow to “fix” thymus function postnatally. Hayes writes: “For the first time physicians are challenged by clinical states in which the T cell pool is destroyed postnatally in large numbers of patients. One such state is AIDS; another is the immune damage of cancer chemotherapy. Accordingly, study of postnatal thymic function is now a matter of clinical urgency. Ongoing work may point toward new strategies for repairing a damaged T-cell repertoire” (Haynes and Hale, 1999).

The cell signaling data suggest that CD4 lineage commitment requires a stronger signal than commitment to CD8 cells (Kovats et al., 1999; Pestano et al., 1999). Molecular basis for this difference seems to be the activity of the lck protein tyrosine kinase associated with the CD4 and CD8 co-receptors. The importance of timing and affinity of interaction between TCR and MHC/peptide complex has recently been demonstrated. High affinity interaction of H-Y class I restricted TCR with male dendritic cells caused these cells to develop into CD4 lineage (Martin & Bevan, 1997). None of the studies with TCRs restricted for MHC class I and class II demonstrated peptide specific positive selection in vivo, however. As such, a clear picture of the precise events leading to selection remains elusive.

### SUMMARY OF THE INVENTION

In accordance with the present invention, the inventors now describe a method for specifically enhancing an antigen specific population of T cells. More particularly, the present invention discloses a method of enhancing T cell immunity by promoting de novo production of antigen specific T cells by administering an epitopic peptide to a subject. The method demonstrates and exploits the ability to induce differentiation of T lymphocytes in the thymus, thereby controlling the antigen specificities of the newly generated T cell lymphocytes. As a result of this intervention, a peripheral T lymphocyte population capable of responding to specific antigens is enriched.

Initial evidence of the efficacy of this method was provided in genetically modified mice which demonstrate that the generation of T cells with desirable antigen specificity can be induced in vivo by providing a positively-selecting ligand, as shown in FIGS. 1A and 1B. A particular embodiment of the present invention is illustrated in FIGS. 2A-2C, which compares thymic development in the absence of a selecting ligand to thymic development after introduction of a synthetic selecting ligand. Mutations introduced into experimental animals described herein do not affect the molecular mechanisms of positive selection, but facilitate experimental design by allowing normally rare cells to become the exclusive population of T cells that develop in the thymus. Mice were genetically modified to limit the presentation of the endogenous peptides so a very narrow peptide/MHC repertoire is found in these mice. This ensures that the vast majority of the thymocytes will not develop in these mice because their receptors cannot find appropriate peptide/MHC complex to get positively selected. Mice were used which were devoid of two molecules involved in antigen presentation: H2-DM and invariant chain Ii. The TCR repertoire was further constrained by introducing a transgenic receptor specific for a known antigen and by excluding the rearrangement of endogenous TCR alpha chains.

Because of the lack of the ligand for positive selection, transgenic thymocytes do not develop into mature CD4 single positive cells, but are arrested at the double-positive stage (FIGS. 2A-2C). As a consequence of the impaired thymic development, there are very few peripheral CD4⁺ lymphocytes (FIGS. 3A and 3B). Thymic development of CD4 cells is restored by providing positively selected peptide ligand. In this system, mice expressing pigeon cytochrome C specific TCR with the H2-DM, Ii and TCR chain genes knocked out were injected intraperitoneally with PCC50V54A peptide (FIGS. 2A-2C). The newly selected thymocytes mature in the thymus to single-positive CD4 cells and migrate to peripheral lymphoid organs (FIG. 3B). These T cells are functional and respond to antigenic stimuli (FIG. 7 and FIG. 11H). These data provide evidence that the T cell repertoire in the thymus can be manipulated in vivo resulting in the enrichment of the peripheral pool of T lymphocytes in cells with known antigen specificity. In addition, a category of peptide ligands have been identified with agonist properties that can induce positive selection, but at the same time are unable to induce negative selection at the concentration used. These two properties present useful criteria for the design of other peptides capable of positively selecting T cells with desired antigen specificities. Controlled thymic selection of T cells can thus be used to enhance or regulate the immune response directed against a specific antigen. Further, mice expressing transgenic receptor and devoid of molecules involved in antigen presentation (H2-DM and invariant chain) are a useful tool to study molecular mechanisms of thymic selection.

The present invention therefore relates to a method of specifically enhancing/regulating an antigen specific population of T cells. In a basic embodiment of the invention, this method involves establishing a population of antigen specific T cells in a host comprising administering to the host a formulation comprising a peptide in a manner that the administration results in the presentation of the peptide in the thymus of said host such that the presentation results in the positive selection of thymocytes, thereby facilitating the maturation of the thymocytes to T cells specific for said peptide. Within the context of this method, it is envisioned that the thymocytes may be CD3⁺CD4⁺CD8⁺, and that they mature into T cells that are CD3⁺CD4⁺CD8⁻. It is further envisioned that specific embodiments will employ the administration of the peptide to a host that is immunologically immature. In a further embodiment, it is contemplated that the administered peptide will comprise a T cell epitope. It is envisioned that such a T cell epitope may be specific for an antigen, and specifically a pathogen antigen. One of ordinary skill would be aware of a variety of pathogens to which T cells are effective, although it is nevertheless specifically contemplated that the pathogen may be a virus, a bacteria, a helminth, a protozoa, and the like. In another embodiment specifically contemplated by the inventors, the antigen is a tumor antigen.

In envisioned embodiments of the invention, the claimed formulation may be delivered by any of a number of routes of administration which would ultimately facilitate thymic selection. Nevertheless, it is specifically envisioned that the peptide may be administered by injection, and further that said injection may be intraperitoneal. In a related context, it is contemplated that the peptide may be delivered in a pharmaceutically acceptable carrier or formulation.

In the context of the invention as claimed, it is specifically contemplated that embodiments of the invention will include a subsequent screening step wherein the host to which the peptide is administered is subsequently screened to detect the presence of T cells specific for the administered peptide. It is considered that this screening will generally be performed subsequent to the administration of said peptide to said host, although a prior control screening will generally also be applicable.

Further, therapies are provided for immunological disorders resulting from, for example, the absence of T cells with desired specificities or overt reaction of the existing T cells to self-tissues. It is contemplated to generate T cells specific for microbial or tumor antigens by designing thymic vaccines combined with gene therapy involving modified bone marrow cells expressing TCR genes recognizing, for example, tumor antigens. It is further contemplated that identification of the proper selecting ligands should allow for the generation of cells with immumosuppressive/regulatory properties that could be used in therapies of autoimmune diseases, such as diabetes, rheumatoid arthritis, lupus, and the like. In another aspect of the present invention, induced selection of antigen specific T cells is particularly useful in newborns and small children that have highly efficient thymic selection and a low number of naïve peripheral T cells. A skilled artisan recognizes that veterinary medicine will benefit from the ability to manipulate the generation of the TCR repertoire in animals of economic import (e.g., commerical animals: livestock, fish), animals of recreational import (e.g., wildlife, fish, zoos) and in animals of domestic import (e.g., companion animals: fish, dogs, cats, etc.)

In another embodiment, injection of a purified peptide, in the absence of adjuvant, is used to induce positive selection of functional CD4⁺ T cells in vivo. These cells repopulate peripheral lymphoid organs and are functional (respond to higher concentrations of the selecting peptide). Peptide selected CD4⁺ T cells have a “naïve” surface phenotype and can be discriminated from CD4 T cells that respond to conventional vaccines (FIG. 11G). The newly selected CD4⁺ cells may also have “regulatory” (immunosupressive) properties and these cells can be identified by surface expression of specific markers like CD25, CD44, CD5 or CTLA-4 and by the pattern of produced inhibitory cytokines like TGFβ or IL-10 and others. In a particular embodiment, peptides that positively select CD4⁺ T cells in vivo have agonist properties, but do not induce negative selection. In a specific embodiment, a single peptide injection is sufficient to induce and maintain positive selection of new CD4⁺ in vivo for about two weeks. A skilled artisan recognizes that this finding is useful to tag and identify thymic stromal cells that induce positive selection.

The present invention as described herein also utilizes a screening system designed to test the capacity of the exogenously provided peptide to positively select antigen specific CD4⁺ T cells in vivo. The system utilizes mice that simultaneously lack the expression of invariant chain, H-2M and α chain of TCR (or RAG molecules) and are transgenic for the tested αβTCR. The system preferably allows for the generation of a large number of thymocytes that are in the same, known stage of differentiation. This system is useful for the study of the profiles of gene and protein expression during different stages of positive selection. A skilled artisan also recognizes that the system allows testing of the relationship between selecting and antigenic peptide for the given TCR.

An additional embodiment provides a method for identifying a gene or gene product involved in positive selection of thymocytes comprising (a) providing an non-human mammal whose thymocytes are arrested at CD4⁺/CD8⁺; (b) administering to the animal a selecting peptide; (c) obtaining a sample of mRNA from a thymocyte population at selected time following the administering of the selecting peptide; and (d) identifying mRNA's that are present in said thymocyte population in a greater or lesser abundance than in a similar non-human mammal that has not been administered said selecting peptide. The non-human mammal may be a mouse. The peptide may be administered intraperitoneally. The thymocyte population may be obtained from fractionated or unfractionated thymus. The time following the administering of the selecting peptide may be 2 hours, 4 hours, 6 hours, 8 hours, 10 hours, 12 hours, 16 hours, 20 hours, 24 hours, 36 hours, 48 hours, 72 hours, four days, five days, six days or one week. The step of identifying may comprise amplification of the mRNA, reverse transcription of said mRNA, hybridization of a cDNA or cRNA product to a chip comprising a nucleic acid array, differential display or subtractive hybrization.

In a related embodiment, there is provided a method for identifying a gene or gene product involved in positive selection of thymocytes comprising (a) providing an non-human mammal whose thymocytes are arrested at CD4⁺/CD8⁺; (b) administering to the animal a selecting peptide; (c) obtaining a sample of protein from a thymocyte population at a selected time following the administering of the selecting peptide; and (d) identifying proteins that are present in the thymocyte population in a greater or lesser abundance than in a similar non-human mammal that has not been administered said selecting peptide. The step of identifying may comprise two-dimensional gel electrophoresis, for example, where the protein sample is labeled with one more more dyes and a fluorescent signal from the resulting gel is scanned. Identifying also may comprise mass spectometry, immunologic detection or protein sequencing.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A-1B—FIGS. 1A through 1B illustrate the principle of the intervention in the generation of the T cell receptor repertoire. FIG. 1A shows natural thymic development in the absence of the exogenously provided selecting ligand, and FIG. 1B shows thymic development after introduction of a synthetic selecting ligand.

FIGS. 2A-2C—FIGS. 2A through 2C shows that PCC-specific TCR transgenic thymocytes can be induced to differentiate in vivo to CD4⁺ single positive thymocytes after addition of the positively selecting peptide ligand. The left panels show thymocytes stained with CD4 and CD8 antibodies, whereas the right panels show thymocytes stained with anti Vβ8 and CD69 antibodies. CD69⁺ cells appear as a result of positive selection on the exogenously provided peptide ligand. In FIG. 2A, thymocytes in PCC TCRα⁻ transgenic mice do not differentiate into single positive CD4⁺ cells in mice deficient in H-2M and Ii. In FIG. 2B, thymocytes from PCC Tg TCRα⁻H-2M⁻Ii⁻ mice after one i.p. injection of 50 μg of the positively selecting peptide PCC50V54A differentiate into single positive CD4⁺ cells. Thymocytes were analyzed 24 hours following peptide injection. In FIG. 2C, PCC Tg TCRα⁻H-2M⁻ Ii⁻ thymocytes analyzed 48 hours after one i.p. injection of 50 μg of the positively selecting peptide PCC50V54A. The upper left quadrants show the differences in the number of CD4⁺ thymocytes before (FIG. 2A) and after (FIGS. 2B and 2C) providing synthetic selecting ligand.

FIGS. 3A-3B—FIGS. 3A through 3B demonstrate peripheral lymph node cells which were stained with anti-CD4 and CD8 antibodies. FIG. 3A shows lymph nodes in PCC Tg TCRα⁻H-2M⁻Ii⁻ transgenic mice have very few CD4- cells. In FIG. 3B, TCRα⁻H-2M⁻ and Ii⁻ mice were injected i.p. with the positively selecting peptide ligand PCC50V54A and lymph node cells were analyzed after two weeks. The upper left quadrant shows the increase in the number of CD4⁺ cells that were generated by introduction of the exogenous peptide ligand.

FIGS. 4A-4E—PCC-specific TCR transgenic thymocytes can be induced to differentiate in vivo to CD4⁺ single positive thymocytes after addition of the positively selecting peptide ligand. Left panels show thymocytes stained with CD4 and CD8 antibodies, right panels show thymocytes stained with anti Vβ8 and CD69 antibodies. At least three mice in each group were analysed. FIG. 4A shows that PCC TCRα⁻ thymocytes differentiate into CD4⁺ cells in C57BL6 mice. The total number of thymocytes was 75.7×10⁶±4.9. FIG. 4B shows that thymocytes in PCC TCRα⁻ transgenic mice do not differentiate into single positive CD4⁺ cells in mice deficient in H-2M and Ii. The total number of thymocytes was 162.8×10⁶±63.3. FIG. 4C shows thymocytes from PCC Tg TCRα⁻ (H-2M⁻ Ii⁻ mice after one i.p. injection of 50 μg of the positively selecting peptide PCC50V54A. Thymocytes were analyzed 24 hours after peptide injection. The total number of thymocytes was 161.5×10⁶±54.4. FIG. 4D shows PCC Tg TCRα⁻ H-2M⁻ Ii⁻ thymocytes analyzed 48 hours after one i.p. injection of 50 μg of the positively selecting peptide PCC50V54A. The total number of thymocytes was 142.5×10⁶±44.9.

FIGS. 5A-5F—PCC-specific TCR transgenic thymocytes can be induced to differentiate in vivo to CD4⁺ single positive thymocytes that leave thymus and repopulate peripheral lymphoid organs. Panels show peripheral lymph node cells stained with CD4 and CD8 antibodies. Two or three mice were analysed in each experimental group. FIG. 5A shows PCC TCR-specific lymph node cells in C57BL6 mouse. The total number of lymph node cells was 39.8×10⁶±11.6. FIG. 5B shows peripheral lymph node cell population in TgTCRα⁻H-2M⁻Ii⁻ mice not injected with the selecting peptide. The total number of lymph node cells was 26.2×10⁶±14.4. FIG. 5C shows peripheral lymph node cell population in TgTCRα⁻H-2M⁻Ii⁻ mice analysed two weeks after injected with the selecting peptide. The total number of lymph node cells was 44.1×10⁶±9.8. FIG. 5D shows PCC Tg TCRα⁻ H-2M⁻ Ii⁻ mice that were thymectomized, and injected with 50 μg of the positively selecting peptide PCC50V54A. Lymph node cells were analysed two weeks after injection. The total number of lymph node cells was 11.5×10⁶±1.6. FIG. 5E shows TCRα⁻H-2M⁻ Ii⁻ mice implanted with neonatal thymi from PCC Tg TCRα⁻ H-2M⁻ Ii⁻ mice. Lymph node cells were analysed two weeks after transplantation. The total number of lymph node cells was 10×10⁶±2.4. FIG. 5F shows TCRα⁻ H-2M⁻ Ii⁻ mice implanted with neonatal thymi from PCC Tg TCRα⁻ H-2M⁻ Ii⁻ mice and injected with the selecting peptide. Lymph node cells were analysed two weeks after transplantation. The total number of lymph node cells was 25.5×10⁶±4.2.

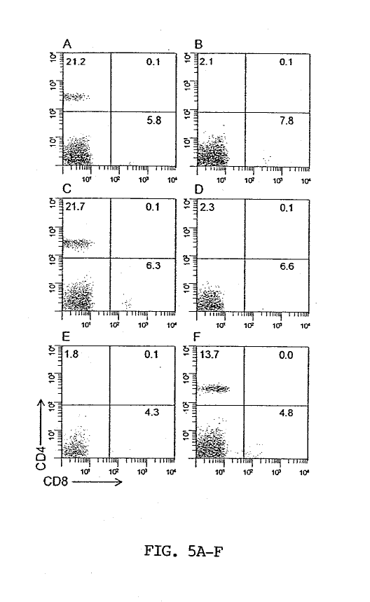

FIG. 6—Lymph node cells from PCC TCRα⁻ Tg mice were stimulated with four agonist peptides that stimulate PCC TCRα⁻ Tg cells that developed on the C57BL6 background—PCC50L, PCC50V, PCC50V54A and PCC50F54A peptides—used at different concentrations, presented by irradiated C57BL6 splenocytes. Proliferation was measured by ³H thymidine incorporation.

FIG. 7—PCC TCR Tg cells selected by the exogenously provided ligand are functional and respond to agonist peptides. Lymph node CD4⁺ cells from PCC TCR Tg and two PCC TgTCRα⁻H-2M⁻Ii⁻ mice (#2 and #12) injected with the selecting peptide PCC50V54A were sorted with magnetic beads and used in the proliferation assay. 50000, 100000 and 200000 responder cells were used. Agonist peptides PCC50V54A and PCC50V at concentration of 20 μM were presented by irradiated splenocytes from C57BL6 mouse. Proliferation was measured by ³H thymidine incorporation. IgGVH peptide was used as a control.

FIG. 8—Neonatal PCC TCR transgenic pups were injected with the selecting peptide just after birth. 24 hours aftr injection pups were killed and thymi were cultured in vitro. After 3-4 days of culture thymocytes were stained with anti CD4 and anti-CD8 antibodies.

FIG. 9—Comparison of the agonist potency of the PCC50V, PCC50L, PCC50V54A, PCC46A49A50VS4A and PCC50F54A peptides. Total lymph node cells from TCR^{Tg}TCRα⁻ mice were stimulated with increasing concentrations of the agonist peptides. Unrelated peptide IgGVH(59-74) was used as a control. The data represent one of two independent experiments.

FIGS. 10A-10F—Development of transgenic cells in TCRα⁻ C57BL6 mice. TCR^{Tg}TCRα⁻ thymocytes were efficiently selected in the thymus expressing wild type A^{b} molecules (FIG. 10A) and repopulate peripheral lymphoid organs (FIG. 10B). The insets in (FIG. 10A) and (FIG. 10B) represent the expression of transgenic Vβ8 chain on CD4⁺ thymocytes and lymph node cells. (FIGS. 10C-10F) The capacity of agonist peptides to induce negative selection of transgenic thymocytes correlates with their agonist potency. Intraperitoneal injection of a moderate agonist peptide PCC50V did not induce negative selection of transgenic thymocytes at a dose of 20 μg and induced only limited deletion at 200 μg/mouse (FIG. 10C, 10E). Injection of the strong agonist peptide PCC50V54A at 20 μg did not induce negative selection but a dose of 200 μg/mouse induced profound deletion of transgenic thymocytes (FIGS. 10D, 10F). Thymocytes and lymph node cells were stained with anti-CD4 and anti-CD8 antibodies and analyzed by flow cytometry. Peptides were injected intraperitoneally at 20 or 200 μg/mouse and mice were sacrificed after 24 hours. The total number of recovered cells is presented above each panel. At least three mice were used in each experiment.

FIGS. 11A-11I. (FIGS. 11A-11D) The development of CD4⁺ T cells in TCR^{Tg}TCRα⁻ H2-M⁻Ii⁻ control mice (FIG. 11A) and mice injected with the selecting peptide PCC50V54A ((FIGS. 11B-11D). Control TCR^{Tg}TCRα⁻H2-M⁻Ii⁻ mice were injected with IgGVH peptide (FIG. 11A). Experimental mice were injected with 20 μg of the PCC50V54A peptide and analyzed after 24 hours (FIG. 11B), 48 hours (FIG. 11C) and 14 days (FIG. 11D). Thymocytes (left panels) and lymph node cells (right panels) were stained with anti CD4 and anti CD8 antibodies and analyzed by flow cytometry. Absolute numbers for thymocytes and lymph node cells in are shown above each panel. (FIG. 11E) Injection of the positively selecting peptide upregulated TCR and CD69 expression on thymocytes. Control mouse (left panel) and mouse injected with the selecting peptide (right panel) were analyzed by flow cytometry 48 hours after peptide administration. (FIG. 11F) Bcl-2 was upregulated in positively-selected thymocytes. TCR^{Tg}TCRα⁻H2-M⁻Ii⁻ mouse was injected i.p. with 20 μg of PCC50V54A peptide and thymocytes were analyzed after 48 hours. Left panel shows thymocyte staining with CD4 and CD8 antibodies and three gates, based on the expression of these two surface markers. Right panel shows bcl-2 expression on gated thymocyte populations I-III. Filled histogram shows staining with isotype-matched control antibody. (FIG. 11G) Peripheral lymph node cells that differentiated in mice injected with the selecting peptide had naïve phenotype. Histograms compare expression of CD44 and CD62L on gated CD4⁺ cells from TCR^{Tg}TCRα⁻ and injected TCR^{Tg}TCRα⁻-H2-M⁻Ii⁻ mice. CD44 expression is depicted by (—) and (-----) and CD62L expression by (--) and (-·-) for cells isolated from TCR^{Tg}TCRα⁻ and injected TCR^{Tg}TCRα⁻ H2-M⁻Ii⁻ mice respectively. (FIG. 11H) The expression of CD2 (LFA-2) and CD11c (LFA-1) was the same on peptide selected CD4⁺ cells as on cells selected in wild type TCR transgenic mice. Histograms compare expression of CD2 and CD11c on gated CD4⁺ cells from TCR^{Tg}TCRα⁻ and injected TCR^{Tg}TCRα⁻H2-M⁻Ii⁻ mice. CD44 expression is depicted by (—) and (-----) and CD1c expression by (--) and (-·-) for cells isolated from TCR^{Tg}TCRα⁻ and injected TCR^{Tg}TCRα⁻H2-M⁻Ii⁻ mice respectively. (FIG. 11I) Peripheral lymph node cells that developed in TCR^{Tg}TCRα⁻ (▴, ▪, ♦) and PCC50V54A injected TCR^{Tg}TCRα⁻H2-M⁻Ii⁻ (Δ, □, ⋄) mice weakly respond in vitro to the selecting peptide PCC50V54A(♦, Δ) or another agonist peptide PCC50V(▪, □). IgGVH (▴, Δ) peptide was used as a negative control.

FIGS. 12A-12B—CD4⁺ cells that appear in injected TCR^{Tg}TCRα⁻H2-M⁻Ii⁻ mice were selected in the thymus. In FIG. 12A, TCR^{Tg}TCRα⁻H2-M⁻Ii⁻ mice were thymectomized and some mice were injected with the selecting peptide. After 14 days the mice were sacrificed and lymph node cells analyzed by flow cytometry. Both control (not injected) (left panel) and injected mice (right panel) had very few CD4⁺ peripheral T cells. In FIG. 12B, TCR^{Tg}TCRα⁻H2-M⁻Ii⁻ thymocytes are selected by the PCC50V54A peptide in thymic graft recipients and repopulate peripheral lymph nodes. Thymi from TCR^{Tg}TCRα⁻H2-M⁻Ii⁻ 2 day old neonates (upper panels) or the same neonates injected with 2 μg of the selecting peptide PCC50V54A (lower panels) were transplanted under kidney capsule of the TCRα⁻H2-M⁻Ii⁻ recipient mice. After 14 days, recipient mice were sacrificed and cells isolated from transplanted thymic tissue (left panels) and recipient lymph nodes (right panels) were stained with anti-CD4 and anti-CD8 antibodies. The absolute number of lymph node cells in control and experimental recipient animals is shown.

FIG. 13—Table lists peptides recognized by transgenic lymphocytes and their biological activities. Total lymph node cells from TCR^{Tg}TCRα⁻ mice were stimulated with increaseing concentrations of the agonist peptides or control peptide—PCC52Q. Graph to the right shows percentage of inhibition of activation (Y axis) with regard to the concentration of the agonist peptides (X axis). Antigen presenting cells were pulsed with agonist peptide PCC50V54A and then used to stimulate transgenic T cells. PCC50E, PCC50N antagonist and PCC52Q neutral peptide were added at concentrations of 0.01, 0.1, 1 and 10 μM. Figure shows percentage of inhibition of antigenic response to PCC50V54A.

FIGS. 14A-14C—The effect of antagonist peptide on thymic selection of TCR^{Tg}TCRα⁻ thymocytes. Antagonist peptides do not induce negative or positive selection of transgenic thymocytes (FIG. 14A). TCR^{Tg}TCRα⁻H2-M⁻Ii⁻ mice were injected i.p. with 50 μg of the antagonist peptide PCC50E. Antagonist peptides inhibit positive selection induced by an agonist selecting ligand (FIGS. 14B & 14C). TCR^{Tg}TCRα⁻H2-M⁻Ii⁻ mice were injected i.p. with 3 μg of the agonist peptide PCC50V54A (FIG. 14A), and mixture of 3 μg of agonist peptide PCC50V54A and 3 μg of antagonist PCC50E (FIG. 14C). Thymocytes were stained with anti CD4 and CD8 antibodies 48 hours after peptide injection. Total thymocyte cell numbers are shown above each panel.

FIGS. 15A-15C. Agonist peptide derived from a mouse natural protein neutral ceramidase has agonist properties and positively selects transgenic thymocytes when injected into TCR^{Tg}TCRα⁻2-M⁻Ii⁻ mouse. (FIG. 15A) Total lymph node cells from TCR^{Tg}TCRα⁻ mice were stimulated with increasing concentrations of PCC50V (▪) or neutral ceramidase derived peptides ( [symbol] ). Unrelated peptide IgGVH(59-74) (♦) was used as a control. Irradiated splenocytes expressing wild type A^{b} were used as APCs. (FIG. 15B) Sorted peripheral lymph node CD4⁺ cells that developed in TCR^{Tg}TCRα⁻ (▪) or TCR^{Tg}TCRα⁻2-M⁻Ii⁻ mice injected with PCCS0V54A (♦) or neutral ceramidase peptide (▴) respond in vitro to the agonist peptide PCC50V. IgGVH peptide was used as a negative control for the response of CD4⁺ cells from TCR^{Tg}TCRα⁻ (□) and TCR^{Tg}TCRα⁻2-M⁻Ii⁻ mice injected with PCC50V54A (Δ) or neutral ceramidase peptide ( [symbol] ). Irradiated splenocytes from H2-M⁻Ii⁻ mice were used as APCs. (FIG. 15C) Neutral ceramidase peptide mediates positive selection of transgenic CD4⁺ thymocytes in TCR^{Tg}TCRα⁻ H2-M⁻Ii⁻ mice. TCR^{Tg}TCRα⁻2-M⁻Ii⁻ mice were injected i.p. with 50 μg of the IgGVH (left panel) and neutral ceramidase (right panel) peptide. The percentage of CD4⁺ single positive cells was 0.8±0.4 and 1.7±0.6 in control and experimental mice 3 days after peptide injection. (FIG. 15D) Positively selected thymocytes repopulated peripheral lymph nodes. Lymph node cells from TCR^{Tg}TCRα⁻2-M⁻Ii⁻ mice were injected with IgGVH (left panel) and neutral ceramidase (right panel) peptide and analyzed 12 days later. The percentage of CD4⁺ positive cells was 2.4±0.8 and 7.6±1.8 in control and experimental mice respectively.

FIGS. 16A-16E—An example of peripheral CD4⁺TCR^{Tg} cells selected by agonist peptide protect mice against an experimental tumor expressing an antigenic complex. B16 melanoma cells were transfected with A^{b}PCC50V54A-YFP construct and 4×10⁶ transfected tumor cells were injected into the TCR^{Tg}α⁻H-2M⁻Ii⁻ mice. Half of the mice received a selecting dose of the PCC50V54A peptide (20 μg, i.p.). After two weeks the number and phenotype of CD4⁺TCR^{Tg} and tumor cells were evaluated in all animals. (FIGS. 16A and 16B) Flow cytometry analysis of draining lymph node cells stained with anti-CD4 and anti-CD8 monoclonal antibodies. (FIGS. 16C and 16D) CD69 and CD62L expression on gated CD4⁺ cells from mice injected only with the selecting peptide (dashed line) or both the selecting peptide and tumor cells (continuous line). Agonist selected CD4⁺ T cells inhibit tumor growth (tumor mass was 4-6 times smaller) and most of the cells that remain in the tumor do not express A^{b}PCC50V54A-YFP (FIG. 16E). Tumor cells from mice injected only with melanoma cells are shown by dashed lines and tumor cells from mice injected with melanoma and the selecting peptide are shown by continuous lines.

FIGS. 17A-17D—Thymocytes expressing the OT-II TCR are positively selected in OT-IITCRα⁻→TCRα⁻H2M⁻Ii⁻ chimeras after injection of a low dose of agonist peptide. OT-II mice express a Class II restricted T cell receptor specific for ovalbumin residues 323-330 in the context of H-2I^{b}. The transgenic OT-II thymocytes are not selected on natural peptides bound to A^{b} in H2-M⁻Ii⁻ chimeras (FIG. 17A). Single injection of 10 μg of Ova(323-339) initiates selection of transgenic CD4⁺Vβ5⁺ thymocytes that continues for 48 hours (FIG. 17B) and 72 hours (FIG. 17C). Freshly selected CD4⁺OT-II⁺ thymocytes start to upregulate the TCR as shown on inserts. These cells also retain their ability to respond to selecting peptide (FIG. 17D). Three mice were used in each experiment and the figures are representative of three experiments.

### DESCRIPTION OF ILLUSTRATIVE EMBODIMENTS

Many studies have analyzed the role peptides play in positive selection of MHC class I and class II restricted T cells. However, despite great effort, these studies yielded conflicting results on the nature of the positively selecting peptide ligand. Development of CD8⁺ thymocytes in fetal thymic organ cultures (FTOCs) from β2-microglobulin or TAP1 knockout mice showed that positive selection can be peptide specific, and that the avidity of the TCR for the MHC/peptide complex determines the fate of immature thymocytes (Tarazona et al., 1998; Sebzda et al., 1996). The response of CD8⁺ thymocytes selected in FTOCs by altered peptide ligands was often compromised (Hogquist et al., 1993; Van Kaer et al., 1992; Hogquist et al., 1995), and none of the selecting peptides identified in vitro supported positive selection of the respective transgenic receptor in vivo (Jameson et al., 1994; Goldrath & Bevan, 1999).

The selection of CD4⁺ T cells in organ cultures has shown that peptide agonists either delete or select class II restricted TCR transgenic thymocytes towards the CD8⁺ lineage (Levelt et al., 1998). Peptide antagonists inhibited the generation of CD4⁺ thymocytes and promoted the selection of CD8⁺ cells or induced negative selection (Kersh et al. 2000; Spain et al., 1994; Volkmann et al., 1998). It also has been reported that agonist ligands can induce differentiation of CD4⁺ thymocytes in reaggregate cultures or in vivo following the intrathymic injection of the recombinant adenovirus encoding the respective peptide (Nikolic-Zugic & Bevan, 1990; Miyazaki et al., 1996; Page et al., 1994). Intrathymic delivery of neopeptides by adenoviral vectors identified antigenic peptides, their analogs without agonist and antagonist activity, and even peptides with unrelated amino acid sequence as being capable of selecting TCRs with defined antigenic specificity.

In contrast, the data described herein indicate that peptides capable of selecting CD4⁺ T cells may have differing primary sequences, but they are required to possess agonist activity. Recent experiments in reaggregation cultures raised the possibility that bone marrow dendritic cells deliver a strong agonist signal necessary to induce CD4⁺ T cell differentiation (Yastumo et al., 2000). Also disclosed by the inventors is the development of transgenic CD4⁺ T cells in chimeras made by reconstituting H-2M⁻Ii⁻ mice with the bone marrow from TCR^{Tg}TCRα⁻ mice (TCR^{Tg}TCRα⁻→H-2M⁻Ii⁻ chimeras). The development of thymocytes in such chimeras is arrested at the CD4⁺CD8⁺ stage despite the presence of selecting wild type A^{b}/self-peptide complexes on bone marrow-derived dendritic cells. Mature CD4⁺ T cells appeared only after injection of the selecting peptide PCC50V54A. This in vivo model relates that, in contrast to the reaggregation cultures (Yastumo et al., 2000), radioresistant thymic epithelial cells are required to provide an agonist signal that induces thymocyte differentiation towards CD4⁺ lineage in vivo.

Positive selection of transgenic thymocytes was induced using a peptide dose that did not delete these cells in wild-type mice. The presentation of injected agonist peptides by wild-type A^{b} molecules present on bone marrow cells in TCR^{Tg}TCRα⁻→H-2M⁻Ii⁻ radiation chimeras also did not induce negative selection. Thus, one could postulate that agonist peptides differ in their relative capacity to induce positive versus negative selection. Some of these peptides induce negative selection even at a very low dose, but others induce positive selection at a low dose and negative selection only when used at a high dose. Under physiological conditions, it is likely that positively selecting peptides are presented at very low concentration so it seems plausible that these peptides would have agonist activity (Wang et al., 1998).

Peptide specific positive selection would favor interaction with a narrow range of more potent agonists, while positive selection of more promiscuous TCRs may involve collective interaction with a broader range of peptides sharing lower agonist potency (Kenty et al., 1998; Nakano et al., 1997). Alternatively, selection of CD4⁺ T cells was achieved using the system described herein because the positively selecting agonist ligand sets the threshold for negative selection, and negative selection requires an interaction with the more potent agonist ligand (Ghendler et al., 1997; Murphy et al., 1990). Signaling studies also support the notion that agonist peptides may be natural ligands for selection of the class II restricted thymocytes (Liblau et al., 1996; Barton & Rudensky, 1999), based on the observation that commitment to the CD4 lineage requires a stronger signal via TCR than commitment towards the CD8 lineage.

Mice transgenic for class II MHC restricted αβTCR and lacking H2-M and Ii molecules likely constitute a non-selecting environment for most of MHC class II restricted transgenic receptors studied so far. The inventors have provided herein the first demonstration that in vivo administration of soluble peptide may restore positive selection of CD4⁺ thymocytes in such mice. This strategy may be used to identify and determine the properties of peptides capable of selecting TCR transgenic CD4⁺ T cells with different antigen specificities in vivo. Since the onset of positive selection of a large number of thymocytes in these studies is known, it is particularly useful for analysis of gene expression patterns associated with positive selection and lineage commitment of CD4⁺ thymocytes within hours after the delivery of the positively selecting signal. Furthermore, the inventors have identified a number of selecting and non-selecting peptides which will make it possible, using soluble recombinant proteins, to determine the relationship between the affinity of the TCR for different peptide/class II MHC complexes and the capacity to induce positive selection in vivo.

It is well known that agonist peptides induce deletion of transgenic thymocytes, although some DP thymocytes were found to be resistant to peptide induced apoptosis (Tarazona et al., 1998; Ghendler et al., 1997; Murphy et al., 1990). However, a number of agonist peptides were shown to induce deletion inefficiently and/or at a very high concentration (Wang et al., 1998; Liblau et al., 1996). The lack of negative selection was attributed to the lower level of class II molecules in H2-M Ii mice, though these molecules are more receptive and present exogenous peptides better than wild-type molecules (Toume et al., 1997; Kenty et al., 1998; Kovats et al., 1999). Thus, in a specific embodiment of the present invention, agonist peptides differ in the relative capacity to induce positive versus negative selection. Some of these peptides induce negative selection even at very low dose, but others induce positive selection at low dose and negative selection only when used at high dose. Positively selecting peptides are presented by the MHC in very low quantity, so it seems plausible that agonist peptides are more likely to deliver sufficient signal to the developing thymocytes (Barton & Rudensky, 1999). Peptide specific positive selection would favor interaction with a narrow range of more potent agonists, while promiscuous positive selection may involve a broader range of peptides with lower agonist potency.

Alternatively, selection of CD4⁺ T cells is possible in the system described herein because the positively selecting agonist ligand sets the threshold for negative selection; hence, negative selection can be achieved only with the more potent agonist ligand (Basu et al., 1998; Grossman & Singer, 1996). Most recently, it was shown that the duration of the thymocyte interaction with the strong agonist ligand determines lineage commitment of thymocytes and longer interaction induces differentiation to CD4⁺ cells in vitro (Yastumo et al., 2000). The signaling studies also support the notion that agonist peptide may be a natural ligand for selection of the CD4 class II restricted thymocytes (Albert Basson et al., 1998; Hernandez-Hoyos et al., 2000).

Regardless of the basis, the experimental system described herein is the first system where positive selection of mature antigen specific CD4⁺ can be induced by a known peptide ligand both in vivo and in vitro. Thus, this system offers the possibility of studying the specificity of interaction between peptide/MHC and TCR that results in positive selection, one of the most debated issues in T cell immunology. In addition, the ability to obtain a large number of cells positively selected at the same time makes it possible to study molecular processes of thymocyte selection, both at the protein and gene expression level.

I. Peptide Epitopes

A. Epitopes

In the context of the invention, a T cell selecting peptide comprises an epitope or eptopic sequence defined as a sequence capable of influencing the thymic maturation of T cells. As to the selection of peptides or polypeptides bearing such an epitope (i.e., that contain a region of a protein molecule to which a TCR can bind), it is well known in that art that relatively short synthetic peptides that mimic a portion of a protein sequence are routinely capable of binding in the context of MHC and being recognized by a TCR. Thus, a peptide or polypeptide comprising one or more epitopic determinants of the T cell selecting peptides of the present invention should generally be at least five or six amino acid residues in length, and may contain up to about 7, 8, 9, 10, 11, 12, 13, 14, 15, 16, 17, 18 19, about 20, or about 25 or so. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing thymic maturation. Preferably, the amino acid sequence of the epitope-bearing peptide is selected to provide substantial solubility in aqueous solvents (i.e., the sequence includes relatively hydrophilic residues and highly hydrophobic sequences are preferably avoided).

Major epitopic determinants of a polypeptide may be identified by an empirical approach in which portions of the gene encoding the polypeptide are expressed in a recombinant host, and/or the resulting proteins tested for their ability to elicit a T cell response. For example, PCR™ can be used to prepare a range of peptides lacking successively longer fragments of the C-terminus of the protein. The immunoactivity of each of these peptides is determined to identify those fragments and/or domains of the polypeptide that are immunodominant. Further studies in which only a small number of amino acids are removed at each iteration then allows the location of the antigenic determinants of the polypeptide to be more precisely determined.

B. Production of Peptides

It is understood that an epitopic composition of the present invention may be made by methods well known in the art, including but not limited to, chemical synthesis by solid phase synthesis and purification away from the other products of the chemical reactions by HPLC. See, for example, Houghten et al. (1985). Preferred methods include synthesis using automated peptide synthesis machines, such as those available from Applied Biosystems (Foster City, Calif.). The epitopic composition may be isolated and extensively dialyzed to remove undesired small molecular weight molecules and/or lyophilized for more ready formulation into a desired vehicle. It is further understood that additional amino acids, mutations, chemical modification and such like, if any, will preferably not substantially interfere with the MHC recognition of the epitopic sequence.

Longer peptides or polypeptides also may be prepared by recombinant means, e.g. by the expression of a nucleic acid sequence encoding a peptide or polypeptide comprising an epitope of the present invention in an in vitro translation system or in a living cell. In certain embodiments, a nucleic acid encoding an antigenic composition and/or a component described herein may be used, for example, to produce an epitopic composition in vitro or in vivo for the various compositions and methods of the present invention. For example, in certain embodiments, a nucleic acid encoding an antigen is comprised in, for example, a vector in a recombinant cell. The nucleic acid may be expressed to produce a peptide or polypeptide comprising an epitopic sequence. The peptide or polypeptide may be secreted from the cell, or comprised as part of or within the cell.

C. Methods of Purifying Peptides

The present invention also provides purified peptides. The term “purified” as used herein, is intended to refer to a proteinaceous composition, wherein the protein material is purified to any degree relative to its naturally-obtainable state, i.e., relative to its purity within a cellular extract or a synthetic chemical mixture. Generally, “purified” also refers to a peptide composition that has been subjected to fractionation to remove various other proteins, polypeptides, or peptides, and which composition substantially retains its activity, as may be assessed, for example, MHC binding and T cell selection, as described herein below, or as would be known to one of ordinary skill in the art.

Where the term “substantially purified” is used, this will refer to a composition in which the specific peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. Various techniques suitable for use in protein purification will be well known to those of skill in the art. The most commonly used separative procedure for chemically synthesized peptides is HPLC chromatography.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding and it should be possible to elute the substance without destroying the sample or the ligand. One of the most common forms of affinity chromatography is immunoaffinity chromatography.

Another example is the purification of a specific fusion protein using a specific binding partner. Such purification methods are routine in the art. As the present invention provides DNA sequences for the specific proteins, any fusion protein purification method can now be practiced. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. However, given many DNA and proteins are known, or may be identified and amplified using the methods described herein, any purification method can now be employed.

II. Vaccines to Improve T Cell Repetoire

A. Vaccination with Peptide Antigens

In accordance with the present invention, one may utilize T cell epitope-containing peptides to select immature T cells from the reservoir of naïve T cells in an immunologically naïve animal. More particularly, the present invention discloses a method of enhancing/regulating T cell immunity by promoting de novo production of antigen specific T cells by administering an epitopic peptide to a subject. The method demonstrates and exploits the ability to induce differentiation of T lymphocytes in the thymus, thereby controlling the antigen specificities of the newly generated T cell lymphocytes.

One establishes a population of antigen specific T cells in a host by administering to the host a formulation comprising a peptide. The manner of administration results in the presentation of the peptide in the thymus of said host such that positive selection of thymocytes occurs, thereby facilitating the maturation of the thymocytes to T cells specific for said peptide. Within the context of this method, it is envisioned that the thymocytes may be CD3⁺CD4⁺CD8⁺, and that they mature into T cells that are CD3⁺CD4⁺CD8⁻. It is further envisioned that specific embodiments will employ the administration of the peptide to a host that is immunologically immature.

B. Genetic Vaccines

In another embodiment, T cell selection is manipulated by inoculating an animal with a nucleic acid encoding a T cell epitope. One or more cells comprised within a target animal then express the sequences encoded by the nucleic acid after administration of the nucleic acid to the animal. Thus, the T cell selecting peptides may comprise a “genetic vaccine” useful for administration protocols. A T cell selecting peptide also may be in the form, for example, of a nucleic acid (e.g., a cDNA or an RNA) encoding all or part of the peptide or polypeptide sequence of an epitope. Expression in vivo by the nucleic acid may be, for example, by a plasmid type vector, a viral vector, or a viral/plasmid construct vector.

In preferred aspects, the nucleic acid comprises a coding region that encodes all or part of a T cell selecting peptide, or an immunologically functional equivalent thereof. Of course, the nucleic acid may comprise and/or encode additional sequences, including but not limited to those comprising one or more immunomodulators or adjuvants. The nucleotide and protein, polypeptide and peptide encoding sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified, combined with the sequences of T cell selecting peptides (e.g., ligated) and/or expressed using the techniques disclosed herein or by any technique that would be known to those of ordinary skill in the art (e.g., Sambrook et al., 1989). Though a nucleic acid may be expressed in an in vitro expression system, in certain embodiments the nucleic acid comprises a vector for in vivo replication and/or expression.

C. Modified Thymic Stromal Cells

In a variation of the embodiments described above, another way of presenting the appropriate ligands to maturing T cells is to use modified thymic stromal cells as a vehicle. In one of these embodiments, thymic epithelial cells are isolated from a subject and transformed with a genetic construct that expresses the peptide ligand of interest. Following selection of ligand-expressing cells and appropriate culturing as needed, these cells are then returned to the subject, where in vivo, these cells express ligand. The second option is to produce thymocytes in vitro using thymic epithelial tumor cell lines expressing the desired selecting ligand. Such cells have been shown to support thymocyte development (Inoue et al., 1998).

III. Delivery and Expression of Peptide Encoding Nucleic Acids

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced, or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. Expression vectors can contain a variety of “control sequences,” which refer to nucleic acid sequences necessary for the transcription and translation of an operably linked coding sequence in a particular host cell. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art. Nonlimiting examples of such regions include the human keratin K14 promoter (Laufer, 1996) #3483) or class II MHC promoter (Kouskoff, 1993) #1644). These two examples of tissue specific promoters limit the expression of the introduced gene to the epithelial or class II MHC positive cells, respectively.

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

5. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

6. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

7. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

8. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

9. Viral Vectors

The ability of certain viruses to infect cells or enter cells via receptor-mediated endocytosis, and express viral genes stably and efficiently have made them attractive candidates for the transfer of foreign nucleic acids into cells (e.g., mammalian cells). Vaccine components of the present invention may be a viral vector that encode one or more peptides. Non-limiting examples of virus vectors that may be used to deliver a nucleic acid of the present invention are described below.

a. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the vaccines of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

c. Retroviral Vectors

Retroviruses have promise as vaccine delivery vectors in due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding a peptide of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

e. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

1. Injection

In certain embodiments, peptides or nucleic acid, may be delivered to an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. The amount of vector used may vary upon the nature of the antigen as well as the organelle, cell, tissue or organism used

2. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

3. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention. In particular, receptors and surface molecules expressed on thymic epithelial cells can be used as targets to specifically deliver the DNA or fusion protein encoding the peptide of interest to this type of thymic stromal cells.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

IV. Pharmaceutical Preparations

Pharmaceutical compositions of the present invention comprise an effective amount of one or more T cell selecting peptides (or nucleic acid encoding therefor), and optional additional agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g. antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The T cell selecting peptides may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). In a particular embodiment, intraperitoneal injection is contemplated.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In nonlimiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The T cell selecting peptides may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In particular embodiments, prolonged absorption of an injectable composition can be brought about by the use in the compositions of agents delaying absorption, such as, for example, aluminum monostearate, gelatin or combinations thereof.

V. Antigens

A. Pathogens

The present invention would have applications therefore in the prevention and treatment of diseases against which antigen-specific and particularly a T cell response would be effective. The following pathogenic virus classes, which are mentioned by way of example, are specifically contemplated as targets for T cell selecting peptide administration: influenza A, B and C, parainfluenza, paramyxoviruses, Newcastle disease virus, respiratory syncytial virus, measles, mumps, parvoviruses, Epstein-Barr virus, rhinoviruses, coxsackieviruses, echoviruses, reoviruses, rhabdoviruses, lymphocytic choriomeningitis, coronavirus, polioviruses, herpes simplex, human immunodeficiency viruses, cytomegaloviruses, papillomaviruses, virus B, varicella-zoster, poxviruses, rubella, rabies, picornaviruses, rotavirus and Kaposi associated herpes virus.

In addition to the viral diseases mentioned above, the present invention is also useful in the prevention, inhibition, or treatment of bacterial infections, including, but not limited to, the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Borellia species, such as Borellia burgedorferi Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species, Haemophilus species, Helicobacter species, including Helicobacter pylori, Treponema species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species, Shigella species, Mycobacterium species (e.g., Mycobacterium tuberculosis, Mycobacterium bovis or other mycobacteria infections), Mycobacterium avium complex (MAC), Mycobacterium marinum, Mycobacterium fortuitum, Mycobacterium kansaii, Yersinia infections (e.g., Yersinia pestis, Yersinia enterocolitica or Yersinia pseudotuberculosis) and the like. In addition, the invention in contemplated to be of use in controlling protozoan, helminth or other macroscopic infections by organisms such as Cryptosporidium, Entamoeba, Plamodiium, Giardia, Leishmania, Trypanasoma, Trichomonas, Naegleria, Isospora belli, Toxoplasma gondii, Trichomonas vaginalis, Wunchereria, Ascaris, Schistosoma species, Cyclospora species, for example, and for Chlamydia trachomatis and other Chlamydia infections such as Chlamydia psittaci, or Chlamydia pneumoniae, for example. Of course it is understood that the invention may be used on any pathogen against which an effective antibody can be made.

Fungal and other mycotic pathogens (some of which are described in Human Mycoses, E. S. Beneke, Upjohn Co.: Kalamazoo, Mich., 1979; Opportunistic Mycoses of Man and Other Animals, J. M. B. Smith, CAB International: Wallingford, UK, 1989; and Scrip's Antifungal Report, by P J B Publications Ltd. 1992) are also contemplated as a target of administration of a T cell selecting peptide. Fungi disease contemplated in the context of the invention include, but are not limited to, Aspergillosis, Black piedra, Candidiasis, Chromomycosis, Cryptococcosis, Onychomycosis, or Otitis externa (otomycosis), Phaeohyphomycosis, Phycomycosis, Pityriasis versicolor, ringworm, Tinea barbae, Tinea capitis, Tinea corporis, Tinea cruris, Tinea favosa, Tinea imbricata, Tinea manuum, Tinea nigra (palmaris), Tinea pedis, Tinea unguium, Torulopsosis, Trichomycosis axillaris, White piedra, and their synonyms, to severe systemic or opportunistic infections, such as, but not limited to, Actinomycosis, Aspergillosis, Candidiasis, Chromomycosis, Coccidioidomycosis, Cryptococcosis, Entomophthoramycosis, Geotrichosis, Histoplasmosis, Mucormycosis, Mycetoma, Nocardiosis, North American Blastomycosis, Paracoccidioidomycosis, Phaeohyphomycosis, Phycomycosis, pneumocystic pneumonia, Pythiosis, Sporotrichosis, and Torulopsosis, and their synonyms, some of which may be fatal. Known fungal and mycotic pathogens include, but are not limited to, Absidia spp., Actinomadura madurae, Actinomyces spp., Allescheria boydii, Alternaria spp., Anthopsis deltoidea, Apophysomyces elegans, Arnium leoporinum, Aspergillus spp., Aureobasidium pullulans, Basidiobolus ranarum, Bipolaris spp., Blastomyces dennatitidis, Candida spp., Cephalosporium spp., Chaetoconidium spp., Chaetomium spp., Cladosporium spp., Coccidioides immitis, Conidiobolus spp., Corynebacterium tenuis, Cryptococcus spp., Cunninghamella bertholletiae, Curvularia spp., Dactylaria spp., Epidermophyton spp., Epidermophyton floccosum, Exserophilum spp., Exophiala spp., Fonsecaea spp., Fusarium spp., Geotrichum spp., Helminthosporium spp., Histoplasma spp., Lecythophora spp., Madurella spp., Malassezia furfur, Microsporum spp., Mucor spp., Mycocentrospora acerina, Nocardia spp., Paracoccidioides brasiliensis, Penicillium spp., Phaeosclera dematioides, Phaeoannellomyces spp., Phialemonium obovatum, Phialophora spp., Phoma spp., Piedraia hortai, Pneumocystis carinii, Pythium insidiosum, Rhinocladiella aquaspersa, Rhizomucor pusillus, Rhizopus spp., Saksenaea vasiformis, Sarcinomyces phaeomuriformis, Sporothrix schenckii, Syncephalastrum racemosum, Taeniolella boppii, Torulopsosis spp., Trichophyton spp., Trichosporon spp., Ulocladium chartarum, Wangiella dermatitidis, Xylohypha spp., Zygomyetes spp. and their synonyms. Other fungi that have pathogenic potential include, but are not limited to, Thermomucor indicae-seudaticae, Radiomyces spp., and other species of known pathogenic genera.

B. Tumor Antigens

In addition, it is specifically contemplated that T cell epitopes derived from tumor antigens may be employed in the context of the invention. Known tumor antigens include, but are not limited to: Adenocorticotropic Hormone (ACTH), Aldosterone, Alphafetoprotein (AFP), Beta-2-Microglobulin (B2M), CA 15-3™, CA 125·, CA 19-₉™, CA 19-9™, CA 549™, Carcinoembryonic Antigen (CEA), p53, Rb, MelanA, HER2/neu, gp100, Ferritin, Gastrin, human Chorionic Gonadotropin (hCG), beta hCG, Gamma Enolase (NSE), Prolactin, Prostatic Acid Phosphatase (PAP), Multiple Melanoma Antigens (MMAs), Prostate Specific Antigen (PSA), Tissue Polypeptide Antigen (TPA), Calcitonin, HOJ-1, estrogen receptor, laminin receptor, erb B, Sialyl Lewis Antigens, tyrosinase, ras, HMFG, -2 and -3, and LD-1.

C. Self-Antigens

In addition to the diseases mentioned above, the present invention is also useful in the prevention, inhibition, or treatment of autoimmune diseases. In this invention, it is specifically contemplated that T cell epitopes derived from body self-proteins may be employed in the context of the invention. Known self antigens include but are not limited to: GAD (glutamic acid decarboxylase), MBP (myelin base protein), Ku protein, thyroglobulin, insulin, acetocholine receptor, snRNP, corticotropin, ATPase proton pump.

VI. Methods for Identifying Thymocyte Selection-Involved Genes

In another embodiment, the present invention provides methods for identifying genes that are involved in the process of thymocyte selection. In particular, these methods involve the use of an experimental animal whose thymocytes are arrested at the CD4⁺CD8⁺ stage. These animals are then stimulated with selecting peptide and expression is monitored at a selected time post-stimulation. The methods may involve examination of nucleic acids or proteins, as described below.

As discussed elsewhere in this document, the inventors have utilized a transgenic animal in which thymocytes are arrested at the CD4⁺CD8⁺ stage. This is the result of alterations in the function of MHC molecules such that the repetoire of antigen that can be presented is severely restricted. In particular, this is achieved by knockout of H2-M and Ii. In addition, a transgenic T cell receptor with known specificity, further limits the repetoire of suitable subtrates. With these two modifications in place, the animal's thymocytes are “stuck” in this developmental stage. Only through the use of a particular peptide can the thymocytes be selected.

A. Methods for Identifying Nucleic Acid Expression

1. Hybridization

Hybridization involves the use of a probe, usually primer between 13 and 100 nucleotides, preferably between 17 and 100 nucleotides in length, or in some aspects of the invention up to 1-2 kilobases or more in length, for creating a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids with appropriate identification means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Specific Methods

In accordance with the present invention, of particular use are methods know as “subtractive hybridization” and “differential display.” An example of the former is U.S. Pat. Nos. 5,436,142 and 5,935,788, which deal with “representational difference analysis,” or RDA. The latter is described by Pardee et al. in U.S. Pat. No. 5,262,311, which is incorporated by reference.

B. Methods for Identifying Protein Expression

1. Immunologic Detection

Any antibody of sufficient selectivity, specificity or affinity may be employed as the basis for immunologic detection. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al, 1991; Silvermann et al., 1995; Cleary et al., 1994; Lenert et al., 1990; Berberianet al., 1993; Kreieretal., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Many appropriate imaging agents are known in the art, as are methods for their attachment to antibodies (see, for e.g., U.S. Pat. No. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (III), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹ , ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium168, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle MH and Ben-Zeev 0, 1999; Gulbis B and Galand P,_{—}1993; De Jager Ret al., 1993; and Nakamura et al., 1987, each incorporated herein by reference.

In general, the immunobinding methods include obtaining a sample suspected of containing ORF expressed message and/or protein, polypeptide and/or peptide, and contacting the sample with a first anti-ORF message and/or anti-ORF translated product antibody in accordance with the present invention, as the case may be, under conditions effective to allow the formation of immunocomplexes.

These methods include methods for purifying an ORF message, protein, polypeptide and/or peptide from organelle, cell, tissue or organism's samples. In these instances, the antibody removes the antigenic ORF message, protein, polypeptide and/or peptide component from a sample. The antibody will preferably be linked to a solid support, such as in the form of a column matrix, and the sample suspected of containing the ORF message, protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the ORF produced antigen, and then detect and quantify the amount of immune complexes formed under the specific conditions.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ORF antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The ORF antigen antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various diseases wherein a specific ORF is expressed, such as an viral ORF of a viral infected cell, tissue or organism; a cancer specific gene product, etc. Here, a biological and/or clinical sample suspected of containing a specific disease associated ORF expression product is used. However, these embodiments also have applications to non-clinical samples, such as in the titering of antigen or antibody samples, for example in the selection of hybridomas.

In the clinical diagnosis and/or monitoring of patients with various forms a disease, such as, for example, cancer, the detection of a cancer specific ORF gene product, and/or an alteration in the levels of a cancer specific gene product, in comparison to the levels in a corresponding biological sample from a normal subject is indicative of a patient with cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Of course, the antibodies of the present invention in any immunodetection or therapy known to one of ordinary skill in the art.

2. Mass Spectometry

Methods for the sequencing of peptides and proteins using mas spectrometry is provide in U.S. Pat. No. 5,952,653, incorporated by reference.

3. Protein Sequencing

The identity of proteins involved in thymocyte selection also may be identified by obtaining samples of protein and performing classic degradative sequencing. The classic approach was first disclosed by Edman & Begg in 1967. Since then, numerous variations employing both amino- and carboxy-terminal approaches have evolved. Hewick et al. (1981); U.S. Pat. Nos. 4,603,114, 5,066,785, 5,270,213 and 5,807,748.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

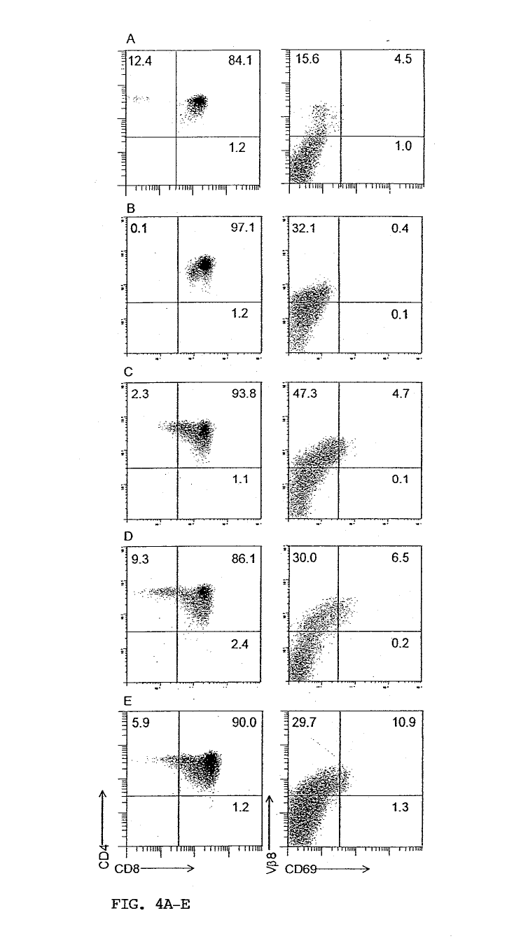

Transgenic mice expressing the αβTCR specific for pigeon cytochrome C derived peptide PCC(43-58) presented by class II MHC molecule A^{b} were generated. This TCR is encoded by the Vα4.5-Jα23Vβ8.1-Dβ2-Jβ2.6 genes and has been expressed as a transgene using the CD2 cassette (Goldrath & Bevan, 1999). The development of CD4⁺ T cells expressing this transgenic TCR proceed normally in C57BL6, A^{bm12} or invariant chain deficient mice A^{b}Ii⁻. In contrast, the maturation of transgenic CD4⁺ T cells was blocked in the thymus in H-2M⁻ mice and in H-2M⁻Ii⁻ double deficient mice, where the A^{b} is occupied with one or very few peptides. (FIGS. 4A-B). In the latter set of mice, thymocytes that express exclusively transgenic PCC TCR (due to the knockout of the endogenous TCRα chain) were arrested at the double positive CD4⁺CD8⁺ stage. The lack of the CD4⁺ single positive cells in these mice was not a result of negative selection as assessed by thymic cellularity, tunnel assay and annexin V staining. Mice without both H-2M and Ii have a low level of class II molecules, they poorly present peptides derived from exogenously supplied proteins, and they have few CD4⁺ T lymphocytes. The resulting narrow repertoire of peptides bound to A^{b} lacks self-peptides capable of positively selecting PCC TCR transgenic cells (Spain et al., 1994; Volkmann et al., 1998; Page et al., 1994; Yastumo et al., 2000; Wang et al., 1998; Nakano et al., 1997).

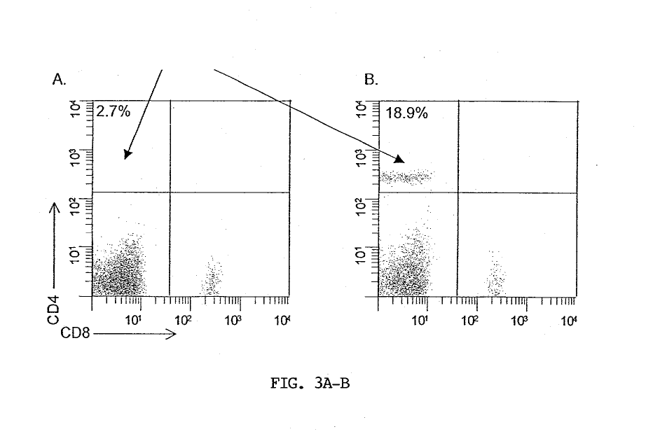

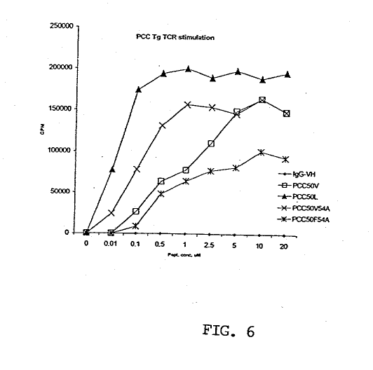

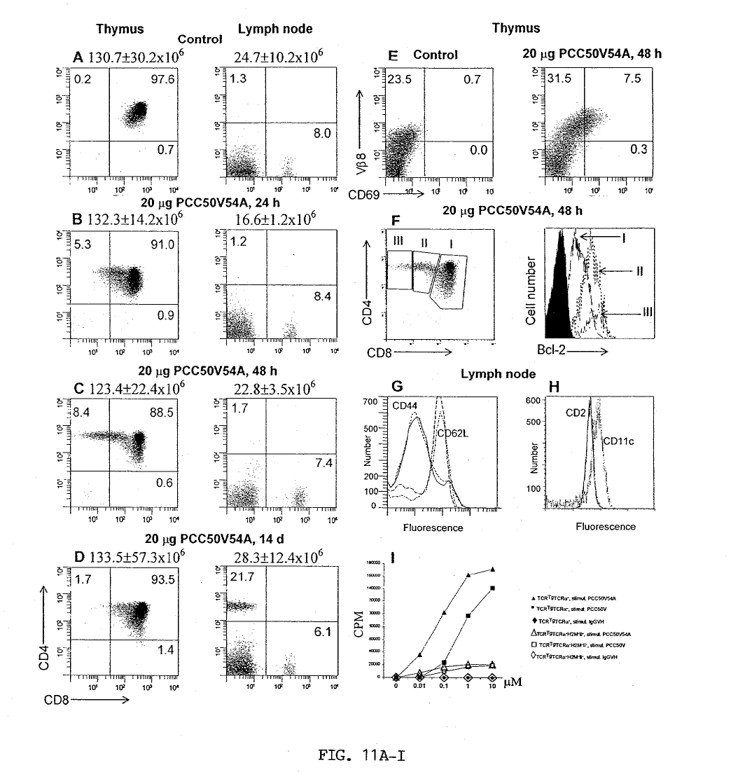

To restore positive selection of CD4⁺ T cells in TgTCRα⁻ H-2M⁻Ii⁻, mice were selected because they express A^{b} molecules occupied with very few endogenous peptides, but these A^{b}molecules efficiently present exogenously added peptides. Since the half-life of the peptides in vivo is short, and the function of CD8⁺ T cells positively selected in vitro by antagonistic peptides is questionable, agonistic peptides that are recognized by this TCR with higher affinity than antagonistic peptides were located. A number of different peptides were tested that bind to A^{b} for example: PCC(43-58), Eαc(52-68), IgGVH (59-74), HEL(81-96), Ova(323-339), Li(8599) and different analogs of these peptides. Among the tested peptides four different peptides were found that activated transgenic CD4⁺ T cells in vitro—(FIG. 6). When these peptides were individually injected i.p. into TgTCRα⁻-2M⁻Ii⁻ mice, all agonist peptides repeatedly induced positive selection of the transgenic CD4⁺ thymocytes, usually starting 24-48 hours after injection (FIG. 3B, FIGS. 4A, 4C, 4D, and 4E and FIGS. 11B-11D).

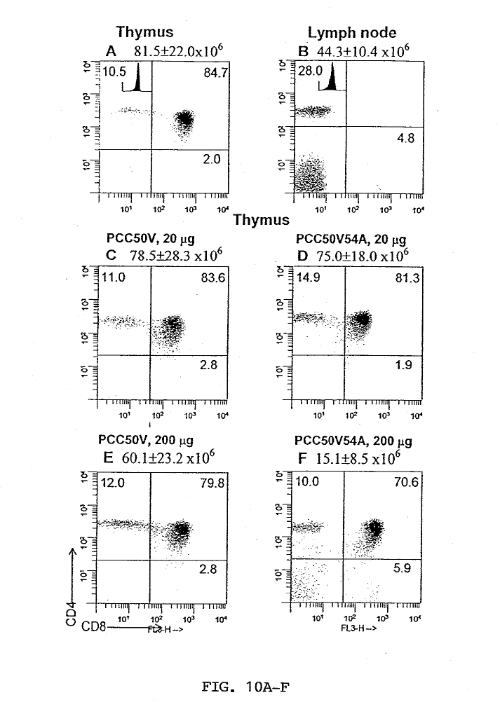

Since both selecting peptides have agonist properties, the induction of negative selection of transgenic thymocytes was investigated. However, thymus cellularity of the TgTCRα⁻H-2M⁻Ii⁻ or TgTCRα⁻A^{b}wt mice injected with 20 μg of the PCC50V54A peptide was the same as in control mice (FIGS. 10A-10F). TUNNEL assay and annexin V staining also failed to demonstrate apoptotic cells after PCC50V54A injection. These results showed that PCC50V54A peptide does not induce negative selection at the tested concentration. Furthermore, staining for gene products induced upon positive selection, e.g., anti apoptotic molecule bcl-2, revealed that positively selected CD4⁺ cells became bcl-2+(FIG. 11F) (Ghendler et al., 1997; Murphy et al., 1990). Accordingly, CD4⁺ T cells can be induced to mature in vivo by a particular category of agonistic peptides that do not induce negative selection.

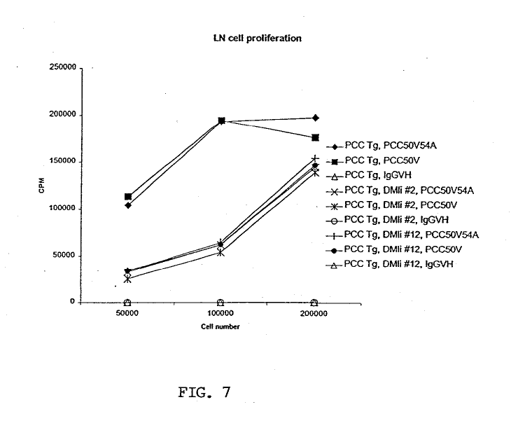

To determine whether the CD4⁺ T cells positively selected by agonistic peptide leave the thymus as functional and mature T cells, the fate of transgenic CD4⁺ cells was followed for two weeks after the peptide was supplied. As shown in FIGS. 3A-B, 5C and 11D, lymph nodes of the TgTCRα⁻ H-2M⁻Ii⁻ mice injected with the peptide had around 8-9×10⁶ CD4⁺ T cells versus 4×10⁵ in the non-injected animals. These CD4⁺ T cells had normal levels of TCR and CD4 and responded to the selecting peptide, although with slightly diminished potency than CD4+transgenic cells selected on unknown self-A^{b}/peptide complex in wild-type mice (FIGS. 7 and 11I). If this phenomenon was derived from the cross-presentation of the same peptide to thymocytes by bone-marrow thymic stromal cells or a result of positive selection by different peptides bound to A^{b} is still unclear. However, these last results showed that mature and functional, CD4⁺ T cells can be positively selected by agonist peptide ligand.

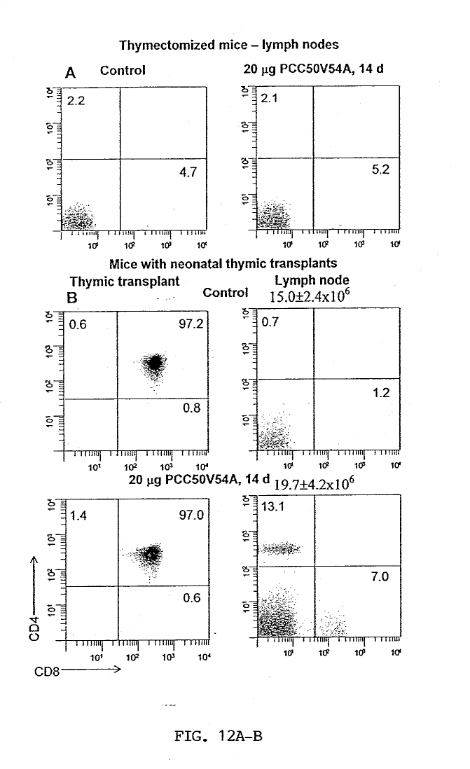

Since the selecting peptide is an agonist, in a specific embodiment of the present invention the observed phenomenon results from the expansion of a small number of peripheral transgenic CD4⁺ T cells rather than from induced positive selection. To test this possibility, two types of experiments have been performed. In the first experiment, the PCC TgTCRα⁻H-2M⁻Ii⁻ mice were thymectomized and then injected with the PCC50V54A peptide. Two weeks after injection, these mice were sacrificed and the number of peripheral CD4⁺ T cells was counted. As shown in FIG. 12A, the number of CD4 transgenic T cells was very low and did not increase in comparison with control mice that were thymectomized, but did not receive the PCC50V54A peptide. In the second experiment, the neonatal thymi from TgTCRα⁻H-2M⁻Ii⁻ mice were transplanted under the kidney capsule of H-2M⁻Ii⁻ TCRα⁻ mice, which are devoid of T cells. Two days after surgery, half of the recipient mice were injected with the selecting peptide. After 10 days, recipient mice were sacrificed and the presence of transgenic CD4⁺ T cells in the transplanted thymus and lymph nodes was determined by FACS analysis. As shown in FIG. 12B transgenic CD4⁺ T cells were found only in thymus-grafted mice, injected with the selecting peptide. Apparently, CD4⁺ T cells appear in the peripheral lymph nodes of PCC TgTCRα⁻H-2M⁻Ii⁻ injected with the PCC50V54A peptide as a result of positive selection by this peptide.

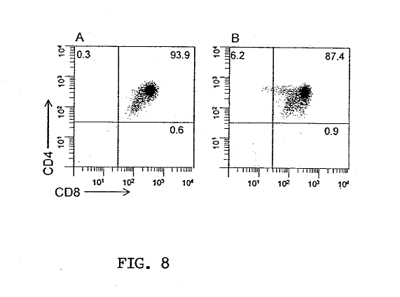

The TgTCRα⁻H-2M⁻Ii⁻ system enables the visualization of thymocyte positive selection in real time and in the native intrathymic environment. An in vitro system was setup where the transgenic thymocytes undergo efficient positive selection and commit to the CD4⁺ lineage. Although continuous delivery of the PCC50V54A peptide at low concentration to FTOCs from the TgTCRα⁻H-2M⁻Ii⁻ mice resulted in positive selection of some transgenic CD4⁺ T cells, the majority of thymocytes committed to the CD8 lineage. It was found that the same proportion of transgenic CD4⁺ T cells differentiated in FTOCs when different concentrations or time of exposure to the peptide was tested. These observations agreed with the previous reports in which CD8⁺, but not CD4+, T cells could be positively selected by the specific peptide in FTOCs (Kenty et al., 1998; Liblau et al, 1996). Therefore, neonatal thymic organ cultures (NTOC) were established from TgTCRα⁻-H-2M⁻Ii⁻ neonates. The neonates received a single i.p. injection of the PCC50V54A peptide just after birth and were sacrificed 24 hours later when their thymuses were harvested. As shown in FIG. 8, the single i.p peptide injection was sufficient to provoke continuous positive selection of a significant number of transgenic CD4⁺ thymocytes in NTOC for the following 4-5 days.

### Example 2

### Materials and Methods

Mice. The TCR genes were cloned from T cell hybridoma specific for analogs of the PCC(43-58) peptide and expressed in VA-hCD2 cassette (Ignatowicz et al., 1997; Kraj et al., 2001; Zhumabekov et al., 1995). All TCR transgenic mice were made by co-microinjection of the respective TCRα⁻ and TCRβ constructs into fertilized eggs of F1 (C57BL/6×CBA/Ca) mice. TCR Tg mice were crossed to C57B16/TCRα⁻ (Jackson Laboratory, ME) mice and to mice deficient in H2-M (kindly provided by E. Bikoff and R. Germain) and Ii (kindly provided by L. van Kaer) to obtain TCR^{Tg}TCRα⁻ H2-M⁻Ii⁻.

Flow cytometry analysis. Monoclonal antibodies specific for CD4(RM4-5), CD8(53-6.7), VβB8(F23.1), CD69(H1.2F3), CD44(IM7) and CD62L(MEL-14) were purchased from PharMingen (San Diego, Calif.) and used according to manufacturer's recommendations. Cells were analyzed using a FACSCalibur instrument (Becton Dickinson, San Jose, Calif.) and CellQuest software. For intracellular stain cells were first stained for CD4 and CD8, fixed in 2% paraformaldehyde, permeabilized in 0.1% Tween-20 and stained with anti-bcl-2 antibody (Pharmingen) according to manufacturer instruction.

Antigen response of TCR transgenic cells. Proliferation of lymph node cells isolated from TCR^{Tg}TCRα⁻and TCR^{Tg}TCRα⁻2-M⁻Ii⁻ mice injected with the selecting peptide was measured in response to antigen. Response of the TCR^{Tg}TCRα⁻ lymph node cells to different agonist peptides was measured by proliferation assay in a 96-well plate. 10⁵ responder cells were stimulated with peptides presented by 5×10⁵ irradiated C57BL6 splenocytes. Agonist peptides were used at concentrations of 0.01, 0.1, 0.5, 1.0, 2.5, 5.0, 10.0 and 20.0 μM. After 3 days cells were pulsed with 1 μCi of [³H]thymidine added to each well and after 18 hours thymidine incorporation was measured. The sequences of peptides used were: PCC50V (AEGFSYTVANKNKGIT), PCC50L (AEGFSYTLANKNKGIT), PCC50V54A (AEGFSYTVANKAKGIT), PCC46A49A50V54A (AEGASYAVANKAKGIT), PCC50F54A (AEGFSYTFANKAKGIT), neutral ceramidase (AGFFQYTLYILASEG) and IgGVH (NADFKTPATLTVDKA). All peptides were synthesized by FMOC (fluorenyl methoxycarbonyl) chemistry and purified by reversed-phase high-performance liquid chromatography. To compare the antigenic response of CD4⁺ TCR Tg cells lymph node cells were collected from TCR^{Tg}TCRα⁻ and TCR^{Tg}TCRα⁻H2-M⁻Ii⁻ mice two weeks after peptide injection. Single cell suspensions were prepared and CD4⁺ cells were sorted using magnetic beads coated with anti CD4 antibodies (MACS). Purity of sorted cells exceeded 85%. 2×10⁴ of purified CD4⁺ cells were used in proliferation assay. Cells were stimulated with increasing concentrations of agonist peptides presented by APCs from wild type or H2-M⁻Ii⁻ mice. IgGVH derived peptide (20 μM) was used as a negative control. Peptides were presented by irradiated splenocytes from a C57BL6 mouse. Proliferation was measured after 3 days by thymidine incorporation by pulsing cells with 1 μCi of [³H] thymidine for 18 hours. Antagonism assay was done by prepulsing 5×10⁵ antigen-presenting cells per well for 2 hr. with 5 μM of the antigenic peptide PCC50V54A, washing, and then exposing them to varying concentrations of peptide analogs with 2×10⁴ purified CD4⁺ TCR^{Tg} lymphocytes as responder cells (Lyons et al., 1996). For antagonism assays the percent of proliferation inhibition was calculated as 100-100*(thymidine incorporation in the presence of antagonist/thymidine incorporation in the absence of antagonist) (Lyons et al., 1996).

Peptide injections and surgical procedures. TCR^{Tg}TCRα⁻ and TCR^{Tg}TCRαH2-M⁻Ii⁻ were injected i.p. with agonist and control peptides dissolved in PBS. After indicated time mice were sacrificed, thymus and lymph nodes were collected and single cell suspensions were prepared. Pups were injected with 2 μg of peptide subcutaneously 12-24 hours after birth and thymi were transplanted into recipient mice 24 hours after injection. 5-8 week old TCR^{Tg}TCRα⁻ H2-M⁻Ii⁻ mice were used for thymectomy or as thymic transplant recipients. Surgical procedures were performed as described (Coligan et al., 1997). Mice were handled according with the institutional guidance.

Protein sequence analysis. Protein sequence datatbase at the National Center for Biotechnology Information was searched using BLAST, Psi-BLAST and FASTA search algorithms with the protein motif (AF)XX(AT)(VLFI)AXX(AN) as a query.

### Results

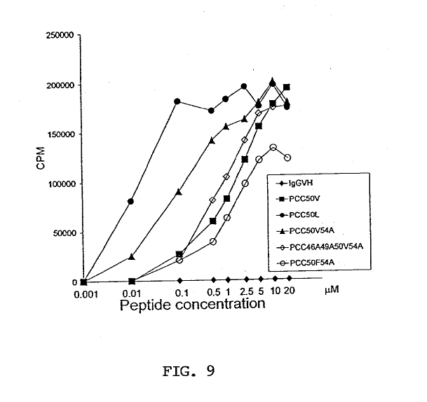

Characterization of peptide agonists for the PCC specific transgenic αβTCR. To examine the role of peptides during positive selection in vivo, the inventors generated transgenic mice expressing class II restricted TCR specific for analogs of a pigeon cytochrome C peptide PCC(43-58) presented by the A molecule (Kraj et al., 2001). This receptor recognizes analogs of PCC(43-58) in which aspartic acid in position 50 is replaced by amino acids with neutral/hydrophobic (PCC50V, PCC50V54A, PCC46A49A50V54A, PCC50L, PCC50F) side chains (FIG. 6 and FIG. 9). The TCR transgenic mice were backcrossed to C57BL6 TCRα⁻ knockout mice so that almost all T cells expressing transgenic TCR become CD4⁺ T cells (FIG. 4A and FIG. 10A). Initially, the inventors examined the capacity of agonist peptides to induce negative selection of transgenic T cells. They found that the efficiency of negative selection correlated with the potency of individual agonist peptides. However, an injection of 20 μg of any tested agonist peptide, in particular a moderate and a strong agonist (PCC50V and PCC50V54A respectively) did not induce negative selection of transgenic thymocytes as assessed by thymus cellularity, annexin V staining and TUNNEL assay (FIGS. 10C and 10D and data not shown). Strong agonists PCC50L and PCC50V54A (FIG. 10F) induced profound negative selection when injected at 200 μg per mouse, while moderate agonist PCC50V induced only marginal deletion of CD4⁺CD8⁺ cells (FIG. 10E).

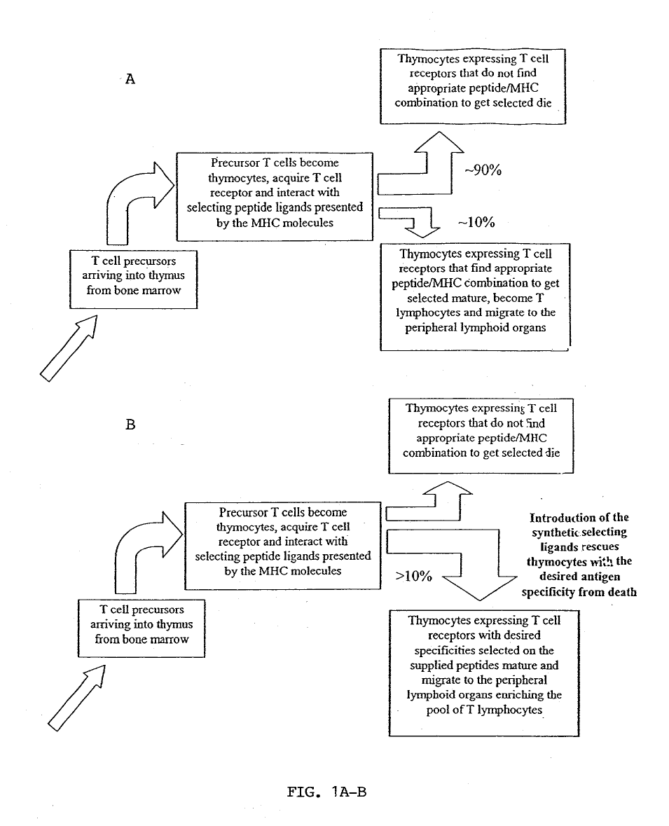

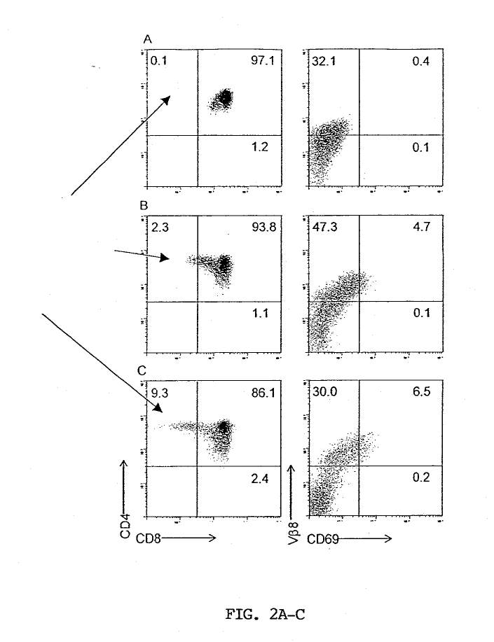

Peptide agonists induce positive selection of transgenic CD4⁺ T cells. To examine the potential effect of agonist peptides on in vivo positive selection of transgenic thymocytes, the inventors followed the ontogeny of these cells in a non-selecting thymic environment where A^{b}molecules are devoid of selecting peptides. For that purpose, the inventors crossed TCR^{Tg}TCRα⁻ mice to mice deficient in invariant chain (Ii) and H2-M to obtain TCR transgenic mice on a triple knockout background (TCR^{Tg}TCRα⁻ H2-M⁻Ii⁻). The development of the majority of CD4⁺ thymocytes is severely impaired in mice lacking H2-M and Ii molecules, two molecular chaperones that participate in peptide loading to class II MHC molecules (Toume et al., 1997; Kenty et al., 1998; Kovats et al., 1998). As expected, the thymic development of the transgenic T cells was arrested at the stage of CD4⁺CD8⁺ thymocytes and only very few transgenic CD4⁺ T cells were detected in the periphery (FIGS. 2A, 4B and FIG. 1A). The lack of the natural positively selecting A^{b}/peptide complex(es) resulted in a block in thymocyte development and increased thymic cellularity in TCR^{Tg}TCRα⁻H2-M⁻Ii⁻ mice. Following these observations, the inventors attempted to restore positive selection in TCR^{Tg}TCRα⁻H2-M⁻Ii⁻ mice by providing exogenous peptides. A number of irrelevant A^{b}-binding peptides (IgGVH(59-74), Ova(323-339), Eα(52-68)) and analogs of PCC (50A, 50N, 50E, 52Q) without agonist properties had no effect on thymic selection (data not shown). As shown in FIGS. 2B-C and 4C-D intraperitoneal injection of a non-deleting dose of PCC50V54A agonist peptide restored selection of CD4⁺ single positive thymocytes. Simultaneously, a number of CD4⁺8⁺ thymocytes upregulated CD69 and bcl-2 expression (FIGS. 11E and 11F). Positive selection of CD4⁺ thymocytes was sustained for 14 days after a single injection of the selecting peptide ligand (FIG. 11D). Contrary to the recent report that agonist peptides induce selection of regulatory CD4⁺CD25⁺ cells in this model newly selected CD4⁺ thymocytes were CD25⁻ (data not shown; Jordan et al., 2001). The cellularity of the thymus and the number of apoptotic cells detected by TUNNEL assay and annexin V staining were the same in controls and in mice injected with 20 μg of agonist peptide PCC50V54A (data not shown). Four other analogs of the PCC peptide, PCC50V, PCC50L, PCC46A49A50V54A and PCC50F54A, injected at the same dose (20 μg), also restored positive selection of transgenic thymocytes (data not shown). These results prove that positive selection of CD4⁺ T cells can be induced in vivo by different agonist ligands. An injection of soluble agonist into TCR^{Tg}TCRα⁻→H2-M⁻Ii⁻ radiation chimeras also resulted in positive selection of transgenic CD4⁺ T cells, despite the expression of wild type A^{b}/peptide complexes on bone marrow derived thymic stromal cells (data not shown).

Positively selected CD4⁺ thymocytes repopulate peripheral lymphoid organs and respond to antigens. To determine whether positively selected CD4⁺ T cells leave the thymus as functional, mature T cells, the inventors analyzed lymph node cells two weeks after peptide administration. As shown in FIG. 1D, TCR^{Tg}TCRα⁻H-2M⁻Ii⁻ mice injected with peptide had approximately 20% CD4⁺ T cells in the lymph nodes when compared to 1-2% CD4⁺ T cells found in control animals (FIG. 11C). Newly selected CD4⁺ T cells had normal levels of TCR and CD4, and the phenotype of naïve T cells (FIGS. 11G and 11H). These cells responded to the selecting peptide, though with lower potency than CD4⁺ transgenic cells isolated from mice expressing wild type A^{b} (FIG. 11I). The reduced response to the selecting peptide might be attributed to the presence of this peptide during thymic development of transgenic T cells.

Since the selecting peptide is an agonist, one could argue that the observed phenomenon results from the expansion of a small number of peripheral transgenic CD4⁺ T cells rather than from induced positive selection (Martin & Bevan, 1997). To test this possibility, two types of experiments were performed. In the first experiment, the TCR^{Tg}TCRα⁻-H-2M⁻Ii⁻ mice were thymectomized and then injected with the PCC50V54A peptide. Two weeks after injection, the mice were sacrificed and the number of peripheral CD4⁺ T cells was counted. As shown in FIG. 12A, the number of CD4 transgenic T cells was very low and did not increase in comparison with control mice that were thymectomized, but did not receive the PCC50V54A peptide. In the second experiment, TCR^{Tg}TCRα⁻H-2M⁻Ii⁻ neonates were injected with the PCC50V54A peptide and after 24 hours thymi from injected and control neonates were transplanted under the kidney capsules of H-2M⁻⁻Ii⁻TCRα⁻ mice, which are devoid of T cells (Takeda et al., 1996). After 10 days, recipient mice were sacrificed and the presence of transgenic CD4⁺ T cells in the transplanted thymus and host lymph nodes was determined by FACS analysis. As shown in FIG. 12B transgenic CD4⁺ T cells were found only in mice grafted with thymi from neonates injected with the selecting peptide. Therefore, the inventors conclude that CD4⁺ T cells appear in the peripheral lymph nodes of TCR^{Tg}TCRα⁻H-2M⁻Ii⁻ injected with the agonist peptide as a result of positive selection by this peptide.

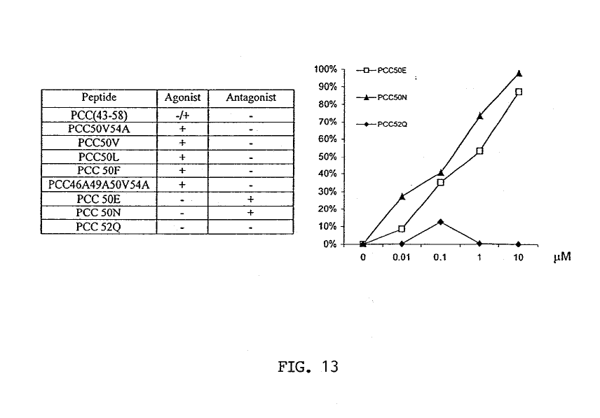

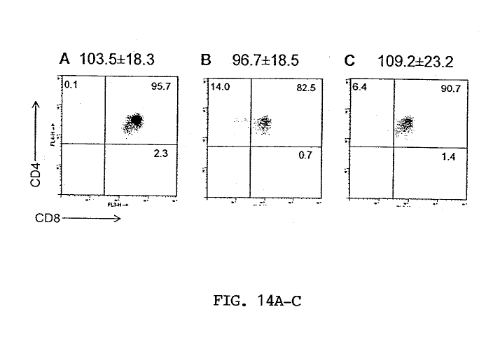

Antagonist peptides do not induce positive or negative selection but inhibit positive selection induced by agonist selecting ligands. It has been shown that the peptide component of the TCR/MHC/peptide complex influences the strength of interaction between a thymocyte and thymic stromal cell and hence determines the fate of the thymocyte (Williams et al., 1997). Minor alterations in critical amino acid residues that are exposed towards the TCR may have a profound effect on the outcome of recognition of MHC/peptide complex by the T cell and change the activation properties of a peptide ligand from agonist to an antagonist (De Magistris et al., 1992). Peptide ligands positively selecting CD8⁺ thymocytes were initially described as having antagonist properties. Later experiments however showed that CD8⁺ thymocytes expressing transgenic TCR were selected by peptides with different activation potencies (Hogquist et al., 1997; Hu et al., 1997). Similar experiments investigating thymocytes expressing class II MHC restricted TCRs in FTOCs, showed that antagonist peptides either inhibit thymic positive selection or induce negative selection (Page et al., 1994; Spain et al., 1994). The inventors have investigated what is the capacity of antagonist peptides to influence thymic selection using this in vivo model. As shown in FIG. 13, the have initially found that analogs of PCC(43-58) with p5 position occupied by E or N have an antagonistic effect on transgenic CD4⁺ T cells. The responses of transgenic CD4⁺ T cells to agonist peptide PCC50A54A were reduced by more than 50% in the presence of each of these analogs, which is the criteria used to identify a given peptide as antagonist (De Magistris et al., 1992). Subsequently, various doses of PCC50E and PCC50N were tested for their ability to affect thymic selection in TCR^{Tg}H2-M⁻Ii⁻ mice. Regardless of wide range of administered doses (up to 50 μg), injection of the antagonist peptides did not result in increased number of CD4⁺ transgenic T cells in the thymus (FIG. 14A) nor induced deletion of CD4⁺CD8⁺ thymocytes. In conclusion, contrary to the “weak affinity” peptide ligands that mediate positive selection of CD8⁺ T cells, the inventors have found that only agonist not antagonist peptides mediate in vivo positive selection of CD4⁺ T cells.

A single thymocyte is likely exposed to numerous different peptides bound to MHC and it has been proposed that the sum of signals produced by these interactions determines thymocyte fate. It was shown that antagonist peptides can affect thymocyte selection, however these results were controversial. In one study antagonist peptide inhibited negative selection of thymocytes while in another study the opposite result was reported (Page et al., 1994; Spain et al., 1994). In addition, an antagonist peptide was also described as being capable of blocking positive selection of CD4⁺ TCR-transgenic thymocytes, although it could not be ruled out that this peptide instead induced late deletion at the stage of single positive cells (Williams et al., 1998). To examine how the antagonist peptide affects positive selection, the inventors co-injected antagonist peptide PCC50E together with the selecting agonist peptide PCC50V54A at the same low dose (3 μg), and evaluated the outcome of selection four days later. As shown in FIGS. 14B and 14C the number of CD4⁺ thymocytes which were positively selected by PCC50V54A agonist peptide was significantly reduced. This effect was also reproduced for the second antagonist peptide PCC50N. It is unlikely that the reduced number of CD4⁺ thymocytes in mice injected with agonist/antagonist mixture is a result of late negative selection because a much higher dose of the same antagonists administered alone did not induce negative selection of CD4⁺CD8⁺ thymocytes. This result argues that a single thymocyte accumulates signals received by interaction with different MHC/peptide complexes.

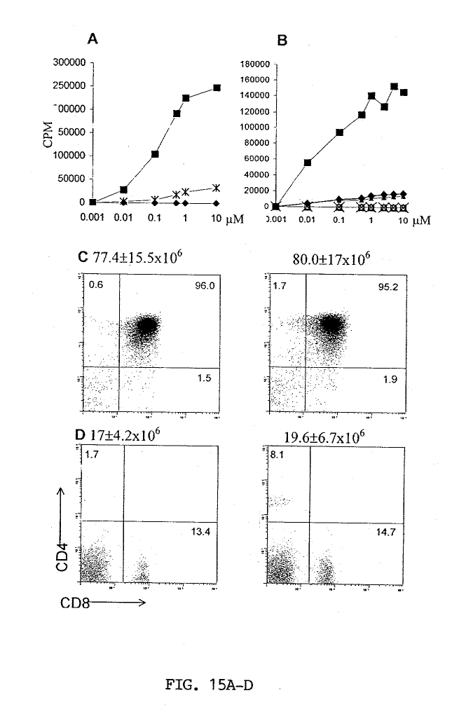

Identification of a candidate natural peptide that mediates positive selection of TCR^{Tg} thymocytes. Since the sequence of the exogenous selecting peptide(s) have been determined, the inventors hypothesized that the natural selecting ligand exists that has agonist properties. By testing different PCC analogs, the inventors have determined that amino acids A or F in position 46, A or T in position 49, A in position 51 and A or N in position 54 are important for binding to A^{b}. Moreover, aminoacids V, L, F, I in position 50 were important to stimulate TCR^{Tg} lymphocytes. The inventors have used the peptide motif (AF)XX(AT)(VLFI)AXX(AN) to search the non-redundant protein and EST protein databases using different computer algorithms. These searches resulted in identification of a mouse proteins which may encode a natural peptides with agonist properties for TCR. One of these proteins was the neutral ceramidase that contained amino acid motif FXXTLYXXA where only Y does not match the original motif (Tani et al., 2000). This protein is ubiquitously expressed in many tissues, including epithelial cells. Subsequently, the inventors synthesized the peptide AGFFQYTLYILASEG which contained the homologous motif sequence. This peptide when tested in vitro acted as weak agonist eliciting proliferation of naïve TCR^{Tg} CD4⁺ T cells (FIG. 15A). Injection of 50 μg of neutral ceramidase peptide into TCR^{Tg}α⁻H2-M⁻Ii⁻ mice resulted in weak positive selection of transgenic CD4⁺ thymocytes (FIG. 15C) and the appeatence of CD4⁺ lymphocytes in the peripheral lymph nodes (FIG. 15D). These peripheral CD4⁺ cells specifically weakly proliferated when stimulted with agonist peptide PCC50V (FIG. 15B). Hence, using biocomputing analysis, the inventors have been able to identify a candidate natural agonist peptide which after injection into TCR^{Tg}H2-M⁻Ii⁻ mice induces positive selection of CD4⁺ T cells. These results imply that the natural peptides bound to class II MHC that select thymocytes are recognized by the relevant TCRs with higher affinity than have been previously postulated.

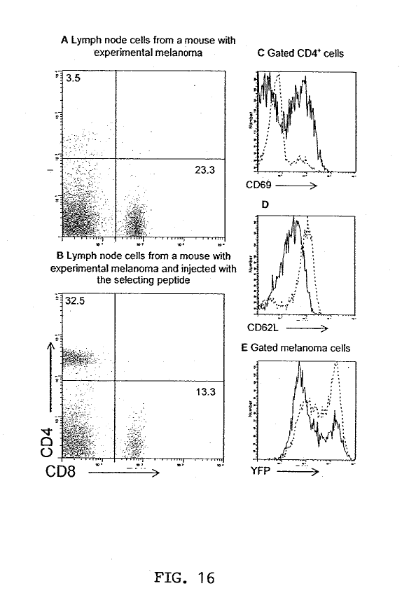

Protection of a mouse melanoma model. Peptide selected CD4⁺ T cells are shown to be functional and protect mice from melanoma tumor cells. To determine if peptide selected T lymphocytes are able to mount an effective immune response in vivo the ability of peptide-selected CD4⁺ cells to protect mice from melanoma was assessed. Melanoma B16-F1 was transfected with constructs encoding the A^{b}βPCC50V54A chain tagged with yellow fluorescent protein (YFP) and A^{b}α chain. These transfectants weakly stimulated TCR^{Tg}α⁻ CD4⁺ cells from wild type mice. TCR^{Tg}α⁻H2-M⁻Ii⁻ mice received subcutaneous injection of B16 melanoma transfectants. At the same time, half of these mice also received selecting agonist peptide. After 12 days, animals were sacrificed, and the phenotype of tumor cells and peripheral CD4⁺ T cells in draining lymph nodes was examined. As shown in FIGS. 16A-E, only mice that received selecting peptide accumulated peripheral CD4⁺ T cells. These cells responded to tumor as assessed by upregulation of CD69 and downregulation of CD62L. Most importantly, tumors in mice that received a selecting peptide were much smaller (4-6 times) and were composed of melanoma cells without surface expression of antigenic complex (only about ¼ of cells were YFP⁺ compared to more then ¾ in mice not treated with the selecting peptide). The phenotype of peptide selected TCR^{Tg}CD4⁺ cells in mice with melanoma tumors was very different from peptide selected cells isolated from TCR^{Tg}α⁻H2-M⁻Ii⁻ mice but not primed with melanoma cells, implying that recent thymic emigrants that left the thymus after agonist injection, become activated upon encounter with tumor cells. In conclusion, although TCR^{Tg}CD4+cells proliferated slower in response to TCR stimulation, it was found that these cells are functional both in vitro and in vivo, and do not share phenotypic markers or properties with CD4⁺CD25⁺ regulatory or anergic T cells.

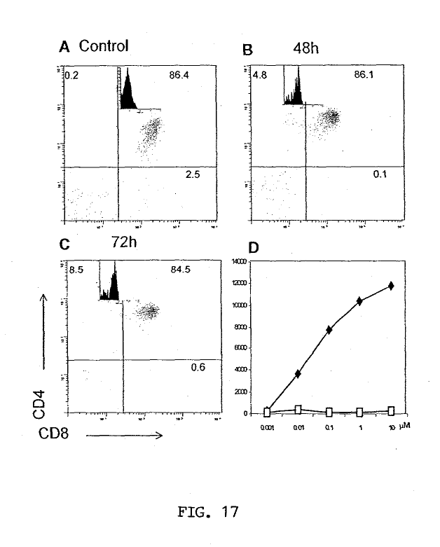

OT-II agonist peptide may be used for positive selection of transgenic CD4⁺ cells. Injection of a low dose of agonist peptides restores positive selection of OT-II transgenic CD4⁺ T cells in OT-II^{Tg}TCRα³¹ →TCRα⁻2-M⁻Ii⁻ chimeras. OT-II transgenic mice (C57BL/6TgN(TcrOva)) express a Class II restricted T cell receptor for ovalbumin residues 323-339 in the context of H-2^{b}. To determine if the experimental approach can be applied to other transgenic CD4⁺ T cells, radiation chimeras were generated where TCRα⁻H2-H2-M⁻Ii⁻ mice were lethally irradiated and reconstituted with fetal liver from OT-II^{Tg}TCRα⁻ mice. In these chimeras the development of OT-II^{Tg}TCRα⁻ thymocytes is arrested at the CD4⁺CD8⁺ stage due to the lack of endogenous selecting peptides. After 8 weeks chimeras were injected once with different doses of the agonist Ova(323-339) peptide, which is a peptide present in the ovalbumin protein. Although this peptide, when injected at a high dose (100 μg and above), induced negative selection of transgenic thymocytes, an injection of a low dose (5-20 μg) induced selection of a significant number of CD4⁺ thymocytes (FIGS. 17A-D). These thymocytes also continued to respond to stimulation by selecting peptide (FIG. 17D). Hence, methods of the present invention may be used to initiate a selection of transgenic CD4⁺ T cells bearing TCR derived from wild type mice.

All of the COMPOSITIONS and/or METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and/or METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,366,241

U.S. Pat. No. 4,472,509

U.S. Pat. No. 4,603,114

U.S. Pat. No. 4,683,195

U.S. Pat. No. 4,683,202

U.S. Pat. No. 4,684,611

U.S. Pat. No. 4,797,368

U.S. Pat. No. 4,800,159

U.S. Pat. No. 4,883,750

U.S. Pat. No. 4,938,948

U.S. Pat. No. 4,952,500

U.S. Pat. No. 5,021,236

U.S. Pat. No. 5,066,785

U.S. Pat. No. 5,139,941

U.S. Pat. No. 5,196,066

U.S. Pat. No. 5,262,311

U.S. Pat. No. 5,270,213

U.S. Pat. No. 5,279,721

U.S. Pat. No. 5,302,523

U.S. Pat. No. 5,322,783

U.S. Pat. No. 5,384,253

U.S. Pat. No. 5,436,142

U.S. Pat. No. 5,464,765

U.S. Pat. No. 5,538,877

U.S. Pat. No. 5,538,880

U.S. Pat. No. 5,563,055

U.S. Pat. No. 5,580,859

U.S. Pat. No. 5,589,466

U.S. Pat. No. 5,591,616

U.S. Pat. No. 5,610,042

U.S. Pat. No. 5,656,610

U.S. Pat. No. 5,702,932

U.S. Pat. No. 5,736,524

U.S. Pat. No. 5,780,448

U.S. Pat. No. 5,789,215

U.S. Pat. No. 5,807,748

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,486

U.S. Pat. No. 5,849,487

U.S. Pat. No. 5,849,497

U.S. Pat. No. 5,849,546

U.S. Pat. No. 5,849,547

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,366

U.S. Pat. No. 5,882,864

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,565

U.S. Pat. No. 5,928,862

U.S. Pat. No. 5,928,869

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,788

U.S. Pat. No. 5,935,791

U.S. Pat. No. 5,935,819

U.S. Pat. No. 5,935,825

U.S. Pat. No. 5,939,291

U.S. Pat. No. 5,942,391

U.S. Pat. No. 5,945,100

U.S. Pat. No. 5,952,653

U.S. Pat. No. 5,981,274

U.S. Pat. No. 5,994,136

U.S. Pat. No. 5,994,624

U.S. Pat. No. 6,013,516

Albert Basson et al., J. Exp. Med. 187, 1249-1260, 1998.

Ashton Rickardt et al., Cell, 76 651-663, 1994.

Atherton et al., Biol. of Reproduction, 32:155-171, 1985.

Ausubel et al., In: Current Protocols in Molecular Biology, John, Wiley and Sons, Inc., 1994.

Baichwal and Sugden, In: Gene transfer, Kucherlapati (ed.), NY Plenum Press, 117-148, 1986.

Barton and Rudensky, Science, 283, 67-70, 1999.

Basu et al., Proc. Nat'l Acad. Sci. USA, 95:14332-14336, 1998.

Bellus, Macromol. Sci. Pure Appl. Chem., A31(1):1355-1376, 1994.

Berberian et al., Science, 261:1588-1591, 1993.

Bickoffet al., J. Exp. Med., 177, 1699-1712 (1993).

Bill and Palmer, Nature, 341, 649-651 (1989).

Blomer et al., J. Virol. 71(9): 6641-6649, 1997

Carbonelli, et al., FEMS Microbiol Lett. 177(1):75-82, 1999.

Chen and Okayama, Mol. Cell. Biol. 7:2745-2752, 1987

Cleary et al., Trends Microbiol., 4:131-136, 1994.

Cocea, Biotechniques, 23:814-816, 1997.

Coligan et al., Current protocols in immunology. Wiley, New York (1997).

Cotten et al., Proc. Natl. Acad. Sci. USA, 89:6094-6098, 1992.

Coupar et al., Gene, 68:1-10, 1988.

Curiel, In: Viruses in Human Gene Therapy, J. -M. H. Vos (Ed.), Carolina Academic Press, Durham, N.C., pp 179-212, 1994.

De Jager R. et al., Semin Nucl Med 23(2):165-179, 1993.

De Magistris et al., Cell 68:625-634 (1992).

Dholakia et al., J. Biol. Chem., 264, 20638-20642, 1989.

Doolittle and Ben-Zeev, Methods Mol Biol., 109:215-237, 1999.

Edman and Begg, Protein Sequenator, European Journal of Biochemistry, 1:80-91, 1967.

EP Appl. 329 822

EPO Patent No. 0273085

Fechheimer et al., Proc Nat'l. Acad. Sci. USA 84:8463-8467, 1987

Fraley et al., Proc Nat'l. Acad. Sci. USA 76:3348-3352, 1979.

Friedmann, “Progress toward human gene therapy,” Science, 244:1275-1281, 1989.

Frohman, In: PCR Protocols: A Guide To Methods And Applications, Academic Press, N.Y., 1990.

GB Appl. 2 202 328

Ghendler et al., Eur. J Immunol., 27, 2279-2289, 1997.

Ghosh and Bachhawat, In: Liver Diseases, Targeted Diagnosis and Therapy Using Specific Receptors and Ligands, Wu and Wu (Eds.), Marcel Dekker, New York, pp 87-104, 1991.

Ginaldi et al., Immunol. Res. 20: 109-115, 1999.

Godthelp et al., Blood 94: 4358-4369 (1999).

Goldrath and Bevan, Immunity, 11, 183-190 (1999).

Gopal, Mol. Cell. Biol. 5:1188-1190, 1985.

Graham and Van Der Eb, Virology 52:456-467, 1973

Grossman and Singer, Proc. Nat'l Acad. Sci. USA, 93:14747-14752, 1996.

Gulbis and Galand, Hum Pathol 24(12):1271-1285, 1993.

Harland and Weintraub, J. Cell Biol. 101:1094-1099, 1985.

Haynes and Hale, Hosp. Pract. (Off Ed) 34: 59-5, 69, 1999.

Hemandez-Hoyos et al., Immunity, 12, 313-322, 2000.

Hogquist et al., Curr. Opin. Immunol., 6, 273-278, 1994a.

Hogquist et al., Cell, 76, 17-27, 1994b.

Hogquist et al., Immunity 6:389-399, 1997.

Hogquist et al., Immunity, 3, 79-86, 1995.

Hogquist et al., J. Exp. Med., 177, 1469-1473, 1993.

Hohler et al., Hum. Immunol. 59: 212-218 (1998).

Horwich et al. J. Virol., 64:642-650, 1990.

Houghten et al., Proc. Nat'l Acad. Sci. USA 82:5131-5135 (1985).

Hu et al., Immunity 7:221-231 (1997).

Inoue et al., Clin. Exp. Immunol. 112: 419-426 (1998).

Jameson et al., Nature, 369, 750-752 (1994).

Kaeppler et al., Plant Cell Reports 9: 415-418, 1990.

Kaneda et al., Science, 243:375-378, 1989.

Kang et al., Science, 240:1034-1036, 1988.

Kato et al., J. Biol Chem., 266(6):3361-3364, 1991.

Kelleher and Vos, Biotechniques, 17(6):1110-1117, 1994.

Kenty et al., J. Immunol., 160:606-614, 1998.

Kersh et al., J. Immunol., 164, 5675-5682 (2000).

Khatoon et al., Ann. of Neurology, 26, 210-219, 1989.

King et al., J. Biol. Chem., 269, 10210-10218, 1989.

Kisielow and von Boehmer, Adv. Immunol. 58, 87-209 (1995).

Kisielow et al., Nature, 333, 742-746 (1988).

Kohler et al., Methods Enzymol., 178:3, 1989.

Kovats et al., J. Exp. Med. 187:245-251, 1998.

Kovats et al., Science, 284, 1187-1191, 1999.

Kraj et al., J. Immunol. 166:2251-2259 (2001).

Kreier et al., In: Infection, Resistance and Immunity,” Harper and Row, New York, 1991.

Kwoh et al., Proc Natl Acad Sci USA. 86(4):1173-1177, 1989.

Laughlin, et al., J. Virol, 60:515-524, 1986.

Lebkowski et al., Mol. Cell. Biol., 8:3988-3996, 1988.

Lenert et al., Science, 248:1639-1643, 1990.

Levelt et al., Proc. Nat'l Acad. Sci. USA, 95, 14349-14354 (1998).

Levenson et al., Human Gene Therapy, 9:1233-1236, 1998.

Liblau et al., Proc. Nat'l Acad. Sci. USA, 93, 3031-3036, 1996.

Macejak and Sarnow, Nature, 353:90-94, 1991.

Mann et al., Cell, 33:153-159, 1983.

Martin and Bevan, Eur. J. Immunol., 27, 2726-2736 (1997).

Martin et al., Cell, 84, 543-550 (1996).

McLaughlin et al., J. Virol., 62:1963-1973, 1988.

Miller, Curr. Top. Microbiol. Immunol., 158:1, 1992.

Miyazaki et al., Cell, 84, 531-541 (1996).

Murphy et al., Science, 250, 1720-1723, 1990.

Muzyczka, Curr. Top. Microbiol. Immunol., 158:97-129, 1992.

Nabel et al., Science, 244:1342-1344, 1989.

Nakamura et al., In: Enzyme Immunoassays: Heterogeneous and Homogeneous Systems, Chapter 27, 1987.

Nakano et al., Science, 275, 678-683, 1997.

Naldini et al., Science, 272(5259):195, 1996

Nicolas and Rubinstein, In: Vectors: A survey of molecular cloning vectors and their uses, Rodriguez and Denhardt (eds.), Stoneham: Butterworth, 494-513, 1988.

Nicolau and Sene, Biochem. Biophys. Acta, 721:185-190, 1982.

Nicolau et al., Methods Enzymol., 149:157-176, 1987.

Nikolic-Zugic and Bevan, Nature, 344, 65-67 (1990).

O'Shannessy et al., J. Immun. Meth., 99, 153-161, 1987.

Owens and Haley, J. Biol. Chem., 259:14843-14848, 1987.

Page et al., Proc. Nat'l Acad. Sci. USA, 91, 4057-4061 (1994).

Paskind et al., Virology, 67:242-248, 1975.

PCT App. PCT/US87/00880

PCT App. PCT/US89/01023

PCT App. WO 94/09699

PCT App. WO 95/96128

PCT App. WO/88/10315

PCT Appl. WO 90/07641

Pelletier and Sonenberg, Nature, 334:320-325, 1988.

Perales et al., Proc. Natl. Acad. Sci. USA, 91(9):4086-4090, 1994.

Pestano et al., Science, 284, 1187-1191 (1999).

Potrykus et al., Mol. Gen. Genet., 199:183-188, 1985.

Potter and Haley, Meth. in Enzymol., 91, 613-633, 1983.

Potter et al., Proc Nat'l Acad. Sci. USA, 81:7161-7165, 1984.

Poulin et al., J. Exp. Med. 190: 479-486 (1999).

Ridgeway, In: Vectors: A survey of molecular cloning vectors and their uses. Rodriguez and Denhardt, eds. Stoneham: Butterworth, pp.467-492, 1988.

Rippe et al., Mol. Cell Biol., 10:689-695, 1990.

Roux et al., Proc. Natl. Acad. Sci. USA, 86:9079-9083, 1989.

Sambrook et al., Molecular Cloning: A Laboratory Manual, 2nd Ed., Cold Spring Harbor Press, Cold Spring Harbor, N.Y., 1989.

Sasso et al., J. Immunol., 142:2778-2783, 1989.

Schelonka et al., Pediatr. Res. 43: 396-402 (1998).

Sebzda et al., Annu. Rev. Immunol. 17, 829-874, 1999.

Sebzda et al., J. Exp. Med., 183, 1093-1104, 1996.

Sha et al., Nature, 336, 73-76 (1988).

Shorki et al., J. Immunol., 146:936-940, 1991.

Silvermann et al., J. Clin. Invest., 96:417-426, 1995.

Spain et al., J. Immunol., 152, 1709-1717 (1994).

Stefanski et al., J. Immunol. 164: 3519-3526 (2000).

Surh and Sprent, Nature, 372, 100-103 (1994).

Tani et al., J. Biol. Chem. 275:11229-11234 (2000).

Tarazona et al., J. Immunol., 160:5397, 5403, 1998.

Temin, In: Gene Transfer, Kucherlapati (ed.), New York: Plenum Press, pp.149-188, 1986.

Tourne et al., Proc. Nat'l Acad. Sci. USA, 94:9255-9260, 1997.

Tratschin et al., Mol. Cell. Biol., 4:2072-2081, 1984.

Tur-Kaspa et al., Mol. Cell Biol., 6:716-718, 1986.

Van Kaer et al., Cell, 71, 1205-1214 (1992).

Volknann et al., J. Exp. Med., 188, 1083-1089, 1998.

Wagner et al., Proc. Nat'l Acad. Sci. USA 87(9):3410-3414, 1990.

Wang et al., Proc. Nat'l Acad. Sci. USA 95, 3804-3809, 1998.

Williams et al., Proc. Natl. Acad. Sci. U.S. A 95:5706-5711, 1998.

Williams et al., Immunol. Today 18:121-126, 1997.

Wilson et al., Science, 244:1344-1346, 1989.

Wong et al., Gene, 10:87-94, 1980.

Wu and Wu, J. Biol. Chem., 262:4429-4432, 1987.

Wu and Wu, Adv. Drug Delivery Rev., 12:159-167, 1993.

Wu and Wu, Biochemistry, 27:887-892, 1988.

Yastumo et al., J. Immunol. 165:3015-3022 (2000).

Zhumabekov et al., J. Immunol. Meth. 185:133-140 (1995).

Zufferey et al., Nat Biotechnol, 15(9):871-875, 1997.

What is claimed is:

1. A method of establishing a population of antigen specific T cells in a host comprising administering to said host a formulation comprising a peptide through a route and in a form that said administration results in the presentation of said peptide in the thymus of said host, said presentation resulting in the positive selection of thymocytes thereby facilitating the maturation of said thymocytes to T cells specific for said peptide.

2. The method of claim 1, wherein said thymocytes are CD3⁺CD4⁺CD8⁺.

3. The method of claim 2, wherein said CD3⁺CD4⁺CD8⁺ thymocytes mature into CD3⁺CD4⁺CD8⁻ T cells.

4. The method of claim 1, wherein said peptide comprises a T cell epitope.

5. The method of claim 4, wherein said T cell epitope is specific for an antigen.

6. The method of claim 5, wherein said antigen is a pathogen antigen.

7. The method of claim 6, wherein said pathogen is a virus.

8. The method of claim 6, wherein said pathogen is a fungus.

9. The method of claim 6, wherein said pathogen is a bacteria.

10. The method of claim 6, wherein said pathogen is a helminth.

11. The method of claim 6, wherein said pathogen is a protozoa.

12. The method of claim 5, wherein said antigen is a tumor antigen.

13. The method of claim 5, wherein said antigen is an autoantigen.

14. The method of claim 1, wherein said formulation is administered by injection.

15. The method of claim 13, wherein said injection is intraperitoneal.

16. The method of claim 1, wherein said formulation is a pharmaceutically acceptable formulation.

17. The method of claim 1, wherein said host is screened for T cells specific for said peptide.

18. The method of claim 17, wherein said screening is subsequent to the administration of said peptide to said host.

19. The method of claim 1, wherein said host is immunologically immature.

20. A method for assessing a test peptide for positively selecting antigen-specific CD4⁺ T cells in vivo comprising:
(a) administering said test peptide to a mouse, wherein said mouse lacks substantial expression of nucleic acid sequences encoding a polypeptide selected from the group consisting of H2-DM, Ii, TCR α, and a combination thereof, and wherein said administering step results in the presentation of said peptide in the thymus of said mouse,
(b) assessing positive selection and maturation of thymocytes to CD4⁺ T cells specific for said peptide.

21. A method for identifying a gene or gene product involved in positive selection of thymocytes comprising:
(a) providing an non-human mammal whose thymocytes are arrested at CD4⁺/CD8⁺;
(b) administering to said animal with a selecting peptide;
(c) obtaining a sample of mRNA from a thymocyte population at selected time following the administering of said selecting peptide; and
(d) identifying mRNA's that are present in said thymocytes population in a greater or lesser abundance than in a similar non-human mammal that has not been administered said selecting peptide.

22. The method of claim 21, wherein said non-human mammal is a mouse.

23. The method of claim 21, wherein said peptide is administered intraperitoneally.

24. The method of claim 21, wherein said thymocyte population is obtained from fractionated or unfractionated thymus.

25. The method of claim 21, wherein the time following the administering of said selecting peptide is 2 hours, 4 hours, 6 hours, 8 hours, 10 hours, 12 hours, 16 hours, 20 hours, 24 hours, 36 hours, 48 hours, 72 hours, four days, five days, six days or one week.

26. The method of claim 21, wherein the step of identifying comprises amplification of said mRNA.

27. The method of claim 21, wherein the step of identifying comprises reverse transcription of said mRNA.

28. The method of claim 27, wherein the step of identifying comprises hybridization of a cDNA or cRNA product to a chip comprising a nucleic acid array.

29. The method of claim 21, wherein the step of identifying comprises differential display.

30. The method of claim 21, wherein the step of identifying comprises subtractive hybrization.

31. A method for identifying a gene or gene product involved in positive selection of thymocytes comprising:
(a) providing an non-human mammal whose thymocytes are arrested at CD4⁺/CD8⁺;
(b) administering to said animal with a selecting peptide;
(c) obtaining a sample of protein from a thymocyte population at selected time following the administering of said selecting peptide; and
(d) identifying proteins that are present in said thymocytes population in a greater or lesser abundance than in a similar non-human mammal that has not been administered said selecting peptide.

32. The method of claim 31, wherein said non-human mammal is a mouse.

33. The method of claim 31, wherein said peptide is administered intraperitoneally.

34. The method of claim 31, wherein said thymocyte population is obtained from fractionated or unfractionated thymus.

35. The method of claim 31, wherein the time following the administering of said selecting peptide is 2 hours, 4 hours, 6 hours, 8 hours, 10 hours, 12 hours, 16 hours, 20 hours, 24 hours, 36 hours, 48 hours, 72 hours, four days, five days, six days or one week.

36. The method of claim 31, wherein the step of identifying comprises two-dimensional gel electrophoresis.

37. The method of claim 36, wherein the protein sample is labeled with one more more dyes and fluorescent signal from the resulting gel is scanned.

38. The method of claim 31, wherein the step of identifying comprises mass spectometry, immunologic detection or protein sequencing.
